(19) United States
(12) Patent Application Publication
(10) Pub. No.: US 2018/0116207 A1
(43) Pub. Date: May 3, 2018

(54) ANTIMICROBIAL POLYMERIC ARTICLES, PROCESSES TO PREPARE THEM AND METHODS OF THEIR USE

(71) Applicant: Johnson & Johnson Vision Care, Inc., Jacksonville, FL (US)

(72) Inventors: Shivkumar Mahadevan, Jacksonville, FL (US); Amit Khanolkar, Jacksonville, FL (US); Osman Rathore, Basking Ridge, NJ (US); Yongcheng Li, St. Augustine, FL (US); Craig W. Walker, Jacksonville, FL (US); Thomas R. Rooney, Jacksonville, FL (US)

(21) Appl. No.: 15/806,832

(22) Filed: Nov. 8, 2017

Related U.S. Application Data
(62) Division of application No. 13/915,657, filed on Jun. 12, 2013, now abandoned.
Application No. 13/915,657 is a division of application No. 11/872,578, filed on Oct. 15, 2007, now abandoned.
(60) Provisional application No. 60/863,628, filed on Oct. 31, 2006.

(51) Int. Cl. A01N 25/00; A01N 25/12; A01N 59/16; A61L 12/08; A61L 27/14; A61L 27/54; G02B 1/04; C08L 83/06

(52) U.S. Cl. CPC A01N 25/00; A01N 25/12; A01N 59/16; A61L 12/088; C08L 83/04; A61L 27/54; G02B 1/043; C08L 83/06; A61L 27/14

(57) ABSTRACT

This invention relates to antimicrobial polymeric articles containing metal salt particles having a particle size of less than about 200 nm dispersed throughout the polymer and methods for their production.

### RELATED APPLICATIONS

This application claims priority from U.S. provisional application Ser. No. 60/863,628, which is incorporated by reference in its entirety.

### FIELD OF THE INVENTION

This invention relates to antimicrobial polymeric articles as well as methods of their production, and use.

### BACKGROUND OF THE INVENTION

Materials with antimicrobial characteristics have been used in many applications. In the case of medical devices such as catheters, prosthetics, implants, ophthalmic devices, surface microbial infestation can result in serious infection and device failure. Surface-centered infections are also implicated in food spoilage, spread of food-borne diseases, and bio-fouling of materials. Hence, there is a significant interest in the development of antimicrobial materials for applications in the health and biomedical device, food, and personal hygiene industries.

Silver salts have a long history of use in human healthcare and medicine as an antiseptic for post surgical infections, in dentistry, wound therapy and medical devices. Silver nitrate has been used for prevention of ophthalmic neonatorum in newborns. Colloidal silver was introduced in 1800s and widely used prior to the 1930s as an alternative to silver nitrate for medical use.

More recently silver compounds have been added to medical devices in various forms, such as soluble and insoluble salts, complexes with binding polymers and zeolites, metallic silver and oxidized silver. However, when many of these silver compounds are incorporated into polymer compositions, the polymer compositions suffer from deficiencies including high haze, inconsistent silver loading, complicated manufacturing, undesirably fast release of the silver, or lack of efficacy.

Several techniques for the incorporation of silver into polymeric matrixes have been disclosed, including chemical workups such as reduction or synthesis of complex silver compounds, mixing preformed silver particles with polymers, or complicated physical techniques such as sputtering and plasma deposition. These processes are complicated and do not always provide consistent loading of the silver compounds in the polymeric materials. The incorporation of oligodynamic metal salts, such as silver salts, as colloidal metal salt particles, into medical devices has been disclosed. However, methods for incorporating said salts into devices formed by photopolymerization, and methods for incorporating said salts into reactive mixtures comprising reducing agents have not been disclosed.

Contact lenses have been used commercially to improve vision since the 1950s. The first contact lenses were made of hard materials. They were used by a patient during waking hours and removed for cleaning. Current developments in the field gave rise to soft contact lenses, which may be worn continuously, for several days or more without removal for cleaning. Although many patients favor these lenses due to their increased comfort, these lenses can cause some adverse reactions to the user. The extended use of the lenses can encourage the buildup of bacteria or other microbes, particularly, Pseudomonas aeruginosa, on the surfaces of soft contact lenses. The build-up of bacteria and other microbes can cause adverse side effects such as contact lens acute red eye and the like. Although the problem of bacteria and other microbes is most often associated with the extended use of soft contact lenses, the build-up of bacteria and other microbes occurs for users of hard contact lens wearers as well.

Therefore, there remains a need to produce ophthalmic devices, such as contact lenses that inhibit the growth of bacteria or other microbes and/or the adhesion of bacterial or other microbes on the surface of ophthlamic devices. Further there is a need to produce ophthalmic devices such as contact lenses that do not promote the adhesion and/or growth of bacteria or other microbes on the surface of the contact lenses. Also there is a need to produce contact lenses that inhibit adverse responses related to the growth of bacteria or other microbes.

### BRIEF DESCRIPTION OF THE DRAWINGS

FIG. 1 is a graph showing silver concentration in the lenses of Example 16 and Comparative Example 2 as a function of distance from the lens edge.

FIG. 2 is a graph comparing the release of silver, as a function of time for the contact lenses made in Examples 16 and Comparative Example 2.

FIG. 3 is a graph comparing efficacy as a function of time for the contact lenses made in Examples 16 and Comparative Example 2 against Pseudomonas aeruginosa.

FIG. 4 shows the UV-VIS spectra for the mixtures of Examples 22 and Synthetic Example 3.

FIG. 5 shows the UV-VIS spectra for the reactive mixtures of Examples 23 A-B.

### SUMMARY OF THE INVENTION

In one embodiment the present invention relates to an article formed from at least one polymer, the polymer comprising, distributed homogeneously throughout, antimicrobial metal salt particles having a particle size of less than about 200 nm, wherein said article displays at least about 0.5 log reduction of at least one of Pseudomonas aeruginosa and s aureus and a haze value of less than about 100% at about 70 microns thickness compared to a CSI lens.

In another embodiment the present invention relates to a process comprising the steps of
(a) dissolving in a solvent at least one salt precursor, optionally with at least one component of a reactive polymer mixture to form a salt precursor mixture;
(b) forming a dispersing agent-metal agent complex by dissolving in a solvent at least one metal agent and at least one dispersing agent, optionally with at least one reactive component to form a metal agent mixture, wherein said solvents and components may be the same or different;
(c) mixing said salt precursor mixture and said metal agent mixture under particle forming conditions to form a particle-containing mixture comprising at least one antimicrobial metal salt, [M^{q+}]ₐ[X^{z-}]_{b};
(d) optionally mixing additional reactive components with said particle containing mixture to form a particle-containing reaction mixture; with the proviso that where no reactive components are included in steps (a) and (b), at least one reactive component is added in step (d); and
   reacting said particle containing reactive mixture to form an antimicrobial polymeric article under reaction conditions sufficient to maintain at least about 90% of M from said metal agent added in step (c) in said polymeric article as M^{q+}.

In yet another embodiment the present invention relates to a process comprising curing a reactive mixture comprising stabilized antimicrobial metal salt particles, having a particle size of about 200 nm or less and at least one free radical reactive component using light of wavelengths above the adjusted critical wavelength for said metal salt particles, heat, or a combination thereof, to form an article comprising antimicrobial metal salt particles.

### DETAILED DESCRIPTION OF THE INVENTION

This invention includes an antimicrobial article that displays at least about 0.5 log reduction of at least one of Pseudomonas aeruginosa, Staphyloccus aureus, or both and a haze value of less than about 100% comprising, consisting essentially of, or consisting of antimicrobial metal salt particles having a particle size of less than about 200 nm dispersed homogeneously throughout at least one polymer from which the article is made. In some embodiments the particle size is less than about 100 nm, and in other embodiments less than about 50 nm. The particles size of the antimicrobial metal salt particles in the article may be measured by scanning electron microscopy.

As used herein, the term, “antimicrobial” means that the article exhibits one or more of the following properties, the inhibition of the adhesion of bacteria or other microbes to the article, the inhibition of the growth of bacteria or other microbes on article, and the killing of bacteria or other microbes on the surface of the article or in an area surrounding the article. For purposes of this invention, adhesion of bacteria or other microbes to the article, the growth of bacteria or other microbes on the article and the presence of bacterial or other microbes on the surface of article are collectively referred to as “microbial colonization.”

Preferably, the articles of the invention exhibit at least about 0.25 log reduction, in some embodiments at least about 0.5 log reduction, and in some embodiments at least about a 1.0 log reduction 90% inhibition) of viable bacteria or other microbes. Such bacteria or other microbes include but are not limited Pseudomonas aeruginosa, Acanthamoeba species, Staphyloccus. aureus, E. coli, Staphyloccus epidermidis, and Serratia marcesens.

Free radical reactive components include polymerizable components which may be polymerized via a free radical initiated reaction. Non-limiting examples of free radical reactive groups include (meth)acrylates, styryls, vinyls, vinyl ethers, C₁₋₆alkyl(meth)acrylates, (meth)acrylamides, C₁₋₆alkyl(meth)acrylamides, N-vinyllactams, N-vinylamides, C₂₋₁₂alkenyls, C₂₋₁₂alkenylphenyls, C₂₋₁₂alkenylnaphthyls, C₂₋₆alkenylphenylC₁₋₆alkyls, O-vinylcarbamates and O-vinylcarbonates.

As use herein, the term “metal salt” means any molecule having the general formula [M^{q+}]ₐ[X^{z-}]_{b}wherein X contains any negatively charged ion, a, b, q and z are independently integers ≥1, q(a)=z(b). M may be any positively charged metal ion selected from, but not limited to, the following Al⁺³, Cr⁺², Cr⁺³, Cd⁻¹, Cd⁺², Co⁺², Co⁺³, Ca⁺², Mg⁺², Ni⁺², Ti⁺², Ti⁺³, Ti⁺⁴, V⁺², V⁺³, V⁺⁵, Sr⁺², Fe⁺², Fe⁺³, Au⁺², Au⁺³, Au⁺¹, Ag⁺², Ag⁺¹, Pd⁺², Pd⁺⁴, Pt⁺², Pt⁺⁴, Cu⁺¹, Cu⁺², Mn⁺², Mn⁺³, Mn⁺⁴, Zn⁺², Se⁺⁴, Se⁺² and mixtures thereof. In another embodiment, M may be selected from Al⁺³, Co⁺², Co⁺³, Ca⁺², Mg⁺², Ni⁺², Ti⁺², Ti⁺³, Ti⁺⁴, V⁺², V⁺³, V⁺⁵, Sr⁺², Fe⁺², Fe⁺³, Au⁺², Au⁺³, Au⁺¹, Ag⁺², Ag⁺¹, Pd⁺², Pd⁺⁴, Pt⁺², Pt⁺⁴, Cu⁺¹, Cu⁺², Mn⁺², Mn⁺³, Mn⁺⁴, Se⁺⁴ and Zn⁺² and mixtures thereof. Examples of X include but are not limited to CO₃⁻², NO₃⁻¹, PO₄⁻³, Cl⁻¹, I⁻¹, Br⁻¹, S⁻², O⁻², acetate, mixtures thereof and the like. Further X includes negatively charged ions containing CO₃⁻², SO₄⁻², POhd 4⁻³, Cl⁻¹, I⁻¹, Br⁻¹, S⁻², O⁻², acetate and the like, such as C₁₋₅alkylCO₂⁻¹. In another embodiment, X may comprise CO₃⁻²SO₄⁻², Cl⁻¹, I⁻¹, Br⁻¹, acetate and mixtures thereof. As used herein the term metal salts does not include zeolites, such as those disclosed in US-2003-0043341-A1. In one embodiment a is 1, 2, or 3. In one embodiment b is 1, 2, or 3. In one embodiment the metals ions are selected from Mg⁺², Zn⁺², Cu⁺¹, Cu⁺², Au⁺², Au⁺³, Au⁺¹, Pd⁺², Pd⁺⁴, Pt⁺², Pt⁺⁴, Ag⁺², and Ag⁺¹ and mixtures thereof. The particularly preferred metal ion is Ag⁺¹. Examples of suitable metal salts include but are not limited to manganese sulfide, zinc oxide, zinc carbonate, calcium sulfate, selenium sulfide, copper iodide, copper sulfide, and copper phosphate. Examples of silver salts include but are not limited to silver carbonate, silver phosphate, silver sulfide, silver chloride, silver bromide, silver iodide, and silver oxide. In one embodiment the metal salt comprises at least one silver salt such as silver iodide, silver chloride, and silver bromide.

In some embodiments of the present invention at least about 90% and in some embodiments at least about 95% of the metal, M, is in the form of metal salt, [M^{q+}]ₐ[X^{z-}]_{b}. The percentage may be calculated from measured values of ionic metal and metal⁰. For example, where the article is a hydrogel contact lens and the antimicrobial metal salt is silver iodide, the ionic metal can be calculated by extracting the lens in phosphate buffered saline solution (Dulbecco's Phosphate Buffered Saline 10× commercially available from MediaTech, Inc. Herndon, Va.), using the procedure described in using USP AppVII until further salt is not present in the extracting solution. After extraction, the article is measured via using instrumental neutron activation analysis (“INAA”). As Ag⁰ is not extractable under the conditions used, all silver measured in the lens after extraction is in Ag⁰ oxidation state.

For embodiments where the article is a medical device in contact with water miscible bodily solutions like blood, urine, tears or saliva, and antimicrobial efficacy of greater than about 12 hours is desired, the metal salt has a Kₛₚ of less than about 2×10⁻¹⁰ in pure water at 25° C. In one embodiment the metal salt has a solubility product constant of not more than about 2.0×10⁻¹⁷ moles/L. In certain embodiments, the article may be a biomedical device, an ophthalmic device or a contact lens.

As used herein, the term “pure” refers to the quality of the water used as defined in the CRC Handbook of Chemistry and Physics, 74th Edition, CRC Press, Boca Raton Fla., 1993. Solubility-product constants (Kₛₚ) measured in pure water at 25° C. for various salts are published in CRC Handbook of Chemistry and Physics, 74th Edition, CRC Press, Boca Raton Fla., 1993. For example, if the metal salt is silver carbonate (Ag₂CO₃), the Kₛₚ is expressed by the following equation

Ag₂CO₃(s)→2Ag+(aq)+CO₃²⁻(aq)

The Kₛₚ is calculated as follows

Kₛₚ=[Ag⁺]²[CO₃²]

As silver carbonate dissolves, there is one carbonate anion in solution for every two silver cations, [CO₃²⁻]=½[Ag⁺]³, and the solubility-product constant equation can be rearranged to solve for the dissolved silver concentration as follows

Kₛₚ=[Ag⁺]²(½[Ag⁺])=½[Ag⁺]³

[Ag⁺]=(2Kₛₚ)^{1/3}

It has been discovered that articles comprising metal salts having solubility product constants of not more than about 2×10⁻¹⁰ when measured at 25° C. will continuously release the metal from lenses for a period of time from one day to thirty days or longer. In one embodiment suitable metal salts comprise silver iodide, silver chloride, silver bromide, and mixtures thereof. In another embodiment the metal salt comprises silver iodide.

The articles of the present invention are made from polymers and may find application in packaging, storage containers and wraps, including packaging for food, drugs and medical devices, biomedical devices, and the like. Biomedical devices include catheters, stents, blood storage bags and tubes, prosthetics, implants, and ophthalmic devices, including ophthalmic lenses (a detailed description of these lenses follows). In one embodiment, the articles of the present invention are made from photopolymerized polymers, and specifically from free radical radical reactive components, such as components polymerized by exposure to visible light. In other embodiment the articles are exposed, during use, to visible and UV light. Such articles include packaging, storage containers, plastic wraps and ophthalmic devices. In one embodiment, the articles of the present invention are ophthalmic devices.

These articles are known in the art and may be formed from a variety of polymers. In some embodiments the article may be formed from one polymer and coated with a different polymer. The antimicrobial polymer may be shaped into the device, or part of the device or used as a coating.

In many of these embodiments the clarity of the article is of concern to users. For example, in one non-limiting embodiment, where the article is an ophthalmic device, such as a contact lens, the very small particles sizes of the metal salt used in the present invention make them particularly suitable. In some embodiments, the present invention has achieved particles sizes of less than about 200 nm, less than about 100 nm and in some embodiments, less than about 50 nm. This very small particle size, smaller than that of visible light wavelengths, makes the articles of the present invention particularly useful for applications where clarity is desired. Such embodiments include, but are not limited to contact lenses, intraocular lenses, blood storage bags and tubing, and food packaging. For applications where optical quality of the polymer is not required, particles bigger than the above range may be used.

In one embodiment, the metal salt particles are also homogeneously distributed throughout the at least one polymer from which the article is made. As used herein “homoegeneously distributed” means that aggregates of particles are not formed and particles are not substantially concentrated in a particular part of the polymer comprising the antimicrobial metal salt. In one embodiment, homogeneously distributed means that there is less than about 20% difference in metal salt particle concentration (measured as weight % based upon the weight of the dry article) between any two regions of the polymer. In another embodiment there is less than about 10% difference in metal salt particle concentration between any two regions, and in yet other embodiments less than about 5% difference in any two regions of the polymer. Uniformity of distribution may be measured in the final article using elemental analysis techniques which use high energy electron to induce emission of characteristic xrays. For this application electron probe microanalysis (EPM) was used (Cameca SX100 and SX50 automated electron microprobes with four wavelength spectrometers using analytical conditions of 20 Kev, 50 nA and 20 um).

In one embodiment, the articles of the present invention are both free from visual haze and undesirable color. Clarity of the antimicrobial article was measured via % haze measured using a sample having a thickness of about 70 microns against a CSI lens described in detail below. Haze values of less than about 100%, less than about 50% may be readily achieved using the present invention.

The color of finished polymer articles may be measured using a spectrophotometer, and reported on CIE 1976 L*a*b* scale. Articles of the present invention may have L* greater than about 89 and in some embodiments greater than about 90, a* less than about 2, in some embodiments less than about 1.4. Color measurements should be made on polymers without polymer components which may effect the color of the final article, such as UV absorbers, handling tints, photochromic compounds, and the like.

The amount of metal salt in the polymer is measured based upon the total weight of the dry polymer. The amount of metal salt in the polymer is dependent upon the end use and end use requirements of the article. For example, in one embodiment where the article is a contact lens, clarity and color are critical. In embodiments where the article is a contact lens and the metal salt is AgI, the amount of silver in the polymer is about 100 ppm to about 1000 ppm, and in some embodiments 200 ppm to about 1000 ppm, based on the dry weight of the polymer. For other embodiments the amount of silver in the polymer may be about 0.00001 weight percent (0.1 ppm) to about 10.0 weight percent, preferably about 0.0001 (1 ppm) to about 1.0 weight percent, most preferably about 0.0001 (1 ppm) to about 0.1 weight percent, based on the dry weight of the polymer. With respect to adding metal salts, the molecular weight of the metal salts determines the conversion of weight percent of metal ion to metal salt, and one of skill in the art can calculate the amount of salt necessary to provide the desired amount of antimicrobial metal.

In one embodiment, the articles of the present invention may be formed by
(a) dissolving at least one salt precursor in at least one component of a reactive polymer mixture to form a salt precursor mixture;
(b) forming a metal agent-dispersing agent complex by dissolving at least one metal agent and at least one dispersing agent in at least one component of the reactive polymer mixture to form a metal agent mixture;
(c) mixing said salt precursor mixture and said metal agent mixture under particle forming conditions to form a particle containing reactive mixture;
(d) optionally mixing additional reactive polymer components with said particle-containing reactive mixture; and
(e) reacting said particle containing reactive mixture to form an antimicrobial polymeric article or part comprising metal salt wherein at least about 90% of the antimicrobial metal, M, is present in the form of metal salt.

The term metal salt has its aforementioned meaning. The term “salt precursor” refers to any compound or composition (including aqueous solutions) that contains a cation that may be substituted with metal ions. In this embodiment, it is preferred that the salt precursor is soluble in lens formulation at about 1 μg/mL or greater. The term does not include zeolites as described US2003/0043341 entitled “Antimicrobial Contact Lenses and Methods of Use,” or activated silver as described in WO02/062402, entitled “Antimicrobial Contact Lenses Containing Activated Silver and Methods for Their Production”. The salt precursor is added to the reactive mixture in at least a stoichiometric amount, and in some embodiments a molar excess, related to the amount of antimicrobial metal desired in the final plastic article. For example, in an embodiment where 20 μg AgI is present in the article as the metal salt, NaI is present in the reactive mixture in an amount of at least about 12 μg. Examples of salt precursors include but are not limited to inorganic molecules such as sodium chloride, sodium iodide, sodium bromide, lithium chloride, lithium sulfide, sodium sulfide, potassium sulfide, sodium tetrachloro argentate, mixtures thereof and the like. Examples of organic molecules include but are not limited to tetra-alkyl ammonium lactate, tetra-alkyl ammonium sulfate, tetra-alkyl phosphonium acetate, tetra-alkyl phosphonium sulfate, quaternary ammonium or phosphonium halides, such as tetra-alkyl ammonium chloride, tetra-alkyl phosphonium chloride, bromide or iodide, and the like. In one embodiment the precursor salt comprises sodium iodide.

The term “metal agent” refers to any composition (including aqueous solutions) containing metal ions. Examples of such compositions include but are not limited to aqueous or organic solutions of silver nitrate, silver triflate, silver acetate, silver tetrafluoroborate, copper nitrate, copper sulfate, magnesium sulfate, zinc sulfate, mixtures thereof and the like. Suitable concentrations of the metal agent in solution can be calculated based upon the desired amount of metal salt to be included in the final article. For example, in one embodiment, the concentration of metal agent is selected to provide about 0.00001 weight percent (0.1 ppm) to about 10.0 weight percent, about 0.0001 (1 ppm) to about 1.0 weight percent, and in another embodiment about 0.0001 (1 ppm) to about 0.1 weight percent, metal salt in the final article.

In some embodiments a stable color is desired. For example, where the plastic article is an ophthalmic device, it may be desirable for the device to have the same color and clarity as the reactive mixture. Silver salts are known to be photosensitive. Therefore, if care is not taken in their formation and the curing of article which contain them, the desired silver salt is not generated in the article. For example, silver iodide is photosensitive to light of wavelengths less than about 400 nm, and if care is not taken, reactive mixtures which are cured via photoinitiation may form undesirably yellow or brown lenses, indicating that the silver salt has been reduced. Photoreduction may be minimized by curing the reaction mixture comprising the metal salt at wavelengths of light that are greater than the wavelength equivalent to the bond energy for the selected metal salt (“critical wavelength”). For example, AgI has a bond energy of 60 kcal/mol. The wavelength associated with this bond energy may be calculated using the electromagnetic equation:

E_{AgI}=hc/(λN_{A})

Where h is Plank's constant, c is the velocity of light, λ is the wavelength of incident radiation and N_{A} is Avagadro's number.

For AgI, λ is 477 nm. Adjustments to the critical wavelength may be made to account for absorption or reflection of energy by mold materials and packaging materials and solutions. So for example, where the article is a contact lens comprising AgI, which is made by direct molding using plastic molds which account for a 10% energy loss in transmission, the adjusted critical wavelength is:

λ=(1-10%)×477 nm

λ=429 nm

Curing conditions in this embodiment therefore include wavelengths above of about 429 nm. Alternatively, the reaction mixture could be cured using conditions which do not include light, such as, but not limited to thermal curing.

Photoreduction can also be minimized by using a molar excess of salt precursor compared to the metal agent so that substantially all of the metal agent is converted to metal salt. Molar ratios of about 1.1:1 or greater salt precursor:metal agent are acceptable. This insures that at least about 90% of the antimicrobial metal, M, in the final article is in the form of metal salt. In some embodiments, the articles are cured using initiators and conditions other than UV light.

At least one of the metal agent mixture and the salt precursor mixture further comprises at least one dispersing agent, and in one embodiment, the metal agent mixture further comprises at least one dispersing agent. Suitable dispersing agents include polymers which comprise functional groups with lone pair electrons. Examples of dispersing agents include hydroxyalkylmethylcellulose polymers, polyvinyl alcohol, polyvinyl pyrrolidone, polyethylene oxide, polysaccharides, such as starch, pectin, gelatin; polyacrylamide, including polydimethylacrylamide, polyacrylic acid, organoalkoxysilanes such as 3-aminopropyltriethoxysilane (APS), methyl-triethoxysilane (MTS), phenyl-trimethoxysilane (PTS), vinyl-triethoxysilane (VTS), and 3-glycidoxypropyltrimethoxysilane (GPS), polyethers, such as polyethylene glycol, polypropylene glycol, boric acid ester of glycerin (BAGE), silicone macromers having molecular weights greater than about 10,000 and comprising groups which increase viscosity, such as hydrogen bonding groups, such as but not limited to hydroxyl groups and urethane groups and mixtures thereof.

In one embodiment the dispersing agent is selected from the group consisting of hydroxyalkylmethylcellulose polymers, polyvinyl alcohol, polyvinyl pyrrolidone, polyethylene oxide, glycerin, boric acid ester of glycerin (BAGE), gelatin and polyacrylic acid, and mixtures thereof. In another embodiment the dispersing agent is selected from the group consisting of hydroxypropylmethylcellulose, polyvinyl alcohol, polyvinyl pyrrolidone, polyethylene oxide, gelatin, glycerin and BAGE and mixtures thereof. In yet another embodiment the dispersing agent is selected from the group consisting of polyvinyl alcohol, polyvinyl pyrrolidone, and polyethylene oxide, and mixtures thereof.

Where the dispersing agent is a polymer, it can have a range of molecular weights. Molecular weights from about 1000 up to several million may be used. The upper limit is bounded only by the solubility of the dispersing agent in the metal salt mixture, the salt precursor mixture and the reactive mixture. For glycoside polymers such as gelatin and methyl cellulose the molecular weight may be above a million. For non-glycoside polymers like polyvinyl alcohol, polyvinyl pyrrolidone and polyacrylic acid, the molecular weight may range from about 2,500 to about 2,000,000, in some embodiments from about 10,000 to about 1,800,000 Daltons, and in other embodiments from about 20,000 to about 1,500,000 Daltons. In some embodiments molecular weights of greater than about 50,000 Daltons may be used, as dispersing agents in this range provide better stabilization in some polymer systems.

Alternatively, the molecular weight of the dispersion-stabilizing polymers can be also expressed by the K-value, based on kinematic viscosity measurements, as described in Encyclopedia of Polymer Science and Engineering, N-Vinyl Amide Polymers, Second edition, Vol 17, pgs. 198-257, John Wiley & Sons Inc. When expressed in this manner, non-glycoside dispersing agent polymers may have K-values from about 5 to about 150, in some embodiments from about 5 to about 100, from about 5 to about 70 and in other embodiments from about 5 to about 50.

When the metal salt nanoparticles are formed directly in a polymer reactive mixture, the dispersing agent may be present in amounts between about 0.001% to about 40 weight %, based upon the weight % of all components in the reactive mixture. In some embodiments the dispersing agent may be present in amounts between about 0.01 weight % and about 30 weight % and in other embodiments between about 0.1 weight % and about 30 weight %. In some embodiments, the dispersing agent is also a reactive component used to form the polymeric article, such as where a contact lens comprising polyvinyl alcohol is produced. In these embodiments the amount of dispersing agent used may be up to about 90 weight % and in some embodiments up to about 100 weight % based upon the weight % of all components in the reactive mixture.

In some embodiments the dispersing agent provides additional benefits to the resulting polymer. For example, where PVP is the particle stabilizing agent, the PVP may provide improvements in wettability, coefficient of friction, water content, mold release and the like, in addition to providing dispersion stabilization. In these embodiments it may be necessary or desirable to include more of the dispersing agent than is necessary to provide dispersion stabilization. In these embodiments, it will be desirable to balance other process conditions such as degassing and ripening steps to insure particles of the desired size are formed.

The salt precursor mixture and metal agent mixture are mixed under particle forming conditions. As used herein, particle forming conditions comprise time, temperature and pH suitable for forming metal salt particles having an average particle size of less than about 200 nm, in some embodiments less than about 100 nm, and in other embodiments less than about 50 nm dispersed throughout the reactive mixture.

The mixing temperature may vary depending upon the reactive components in the reactive mixture. Generally mixing temperatures above the freezing point of the reactive mixture to about 100° C. may be used. In some embodiment mixing temperatures between about 10° C. and about 90° C. may be used, and in others mixing temperatures between about 10° C. and about 50° C. are useful.

Either or both the salt precursor mixture or the metal agent mixture may be degassed prior to mixing with the reactive mixture.

In one embodiment either the salt precursor mixture or the metal agent mixture may be introduced via a stream, such as a single-jet or both may be simultaneously introduced via double-jet. In the single-jet method, the solution, for example the metal agent mixture, is run through a jet at a controlled rate, into a stirred solution containing the salt precursor mixture, and the dispersing agent. Alternatively, a double-jet process may be used to simultaneously add both the metal agent mixture and the salt precursor mixture by two separate jets to a stirred solution containing the dispersing agent. In some embodiments it may be desirable to add further quantities of the dispersing agent, the salt precursor mixture and/or the metal agent mixture.

The salt precursor mixture and metal agent mixtures may be added to the reactive mixture over times of less than about 10 minutes and in some embodiments over addition times of between about 10 seconds and about 5 minutes.

Any mixing time may be used, so long as the resulting solutions are homogenous, and stable dispersions have been formed. As used here, stable dispersions do not substantially settle for at least about 12 hours. Commercially desirable mixing times may include from about one minute to several days, and in some embodiments from about 10 minutes to about 12 hours.

High shear mixing techniques may be used with low MW polymers, and allow for mixing times at the lower end of the ranges listed above.

The reactive mixture may also be degassed under vacuum or using a gas that does not react with any of the components in the reactive mixture. Suitable inert gases include nitrogen, argon, mixtures including them and the like. The degassing may be conducted using pressures up to full vacuum (e.g. 10 mbar) and for times up to about 60 minutes, and in some embodiments up to about 40 minutes. The duration of the degas step, as well as the temperature and pressure to be used with a given reactive mixture may also depend upon other factors, such as the volatility of the solvent used.

The process may further comprises a particle ripening step prior to the degas step. Very small particles dissolve more readily than larger particles, upon heating. Thus, in applications where clarity is important (such as in ophthalmic devices), it may be desirable to include a particle ripening step to insure that the particles are large enough not to ripen excessively during further processing (such as sterilization, melt processing, annealing, sintering) or storage. In the particle ripening step the reactive mixture is heated to temperatures of about 30 to about 70° C. for times from 5 minutes to 1 hour to reduce the fines. This step may be particularly useful for medical devices which are sterilized. For example, where the plastic article is a lens, the lens must be free from visual haze when it is formed and must remain free from visual haze through processing (including packaging sterilization), storage and use. The creation of fines may also be reduced by decreasing the amount of dispersing agent.

In one embodiment, at least 90% of the particles in the reactive mixture have a particle size of less than about 100 nm, in another at least 90% of the particles in the reactive mixture have a particle size of less than about 80 nm, and in yet another embodiment at least 90% of the particles in the reactive mixture have a particle size of less than about 60 nm. The particle size of the particles in the reactive mixture may be measured via light scattering (either laser or dynamic), as described in the test methods section, below.

The particle containing reactive mixtures of the present invention are both free from visual haze and undesirable color. Lack of undesirable color may be evaluated subjectively against a white background or using the L*a*b* method described below.

Additional components may be optionally added during the mixing step. Additional polymer components include, reactive monomers, prepolymers and macromers, initiators, crosslinkers, chain transfer agents, UV absorbers, wetting agents, release aids, photochromic compounds, nutraceutical and pharmaceutical compounds, colorants, dyes, pigments combinations thereof and the like. They may be added in any form, including as monomers, oligomers or prepolymers.

If any component of the reactive mixture is capable of reacting with the metal agent to form elemental metal and the elemental metal is not desired, that component is, in one embodiment, added to the reactive mixture after the metal salt particles have been formed, but prior to curing of the reactive mixture to form the polymeric article. For example, it has been found that AgNO₃ can react with N,N-dimethylacrylamide (DMA) to form unwanted Ag⁰. Accordingly for reactive mixtures comprising DMA, the DMA is in one embodiment (where the metal salt is AgI) added to the reactive mixture after the metal salt particles (AgI) have been formed. Those of skill in the art can readily determine whether a component acts as a reducing agent by mixing the component with the metal agent in a solvent, and analyzing by chemical analysis or, in certain cases, by the change of the visual appearance of the mixture.

Alternatively the nanoparticle metal salt may be formed separate from the polymer reactive mixture. For example, stabilized metal salt particles may be formed by forming a salt precursor solution comprising at least one salt precursor;

forming a metal agent solution comprising between about 20 and about 50 weight % at least one dispersing agent having a weight average molecular weight of at least about 1000 and at least one metal agent;

adding one solution to the other at a rate sufficient to maintain a clear solution throughout addition and to form a product solution comprising stabilized metal salt particles having a particle size of less than about 200 nm; and drying said stabilized metal salt particles. Stabilized metal salt particles are metal salt particles which have a particle size of less than about 200 nm, and which are complexed with at least one dispersing agent. In some embodiments the stabilized metal salt particles have a particle size of less than about 100 nm, and in some embodiments less than about 50 nm.

In this embodiment the metal agent and salt precursor solutions are formed using any solvent which (a) can dissolve the metal agent, salt precursor and dispersing agent, (b) does not reduce the metal agent to metal and (c) can be readily removed by known methods. Water, alcohols or mixtures thereof may be used. Suitable alcohols may be selected which are capable of solubilizing the metal agent and salt precursor. When silver nitrate and sodium iodide are used as the metal agent and salt precursor, alcohols such as t-amyl alcohol, tripropylene glycol monomethyl ether, and mixtures thereof and mixtures with water may be used. Water may also be used alone.

Any of the dispersing agents described above may be used. Mixtures may be used. The dispersing agent may be included in either or both the metal agent and salt precursor solutions, or can be included in a third solution, into which the metal agent and salt precursor solutions are added. In one embodiment the metal agent solution also comprises at least one dispersing agent. In embodiments where both the salt precursor solution and metal agent solutions comprise at least one dispersing agent, the dispersing agents may be the same or different.

The dispersing agent is included in an amount sufficient to provide a metal salt particle size of less than about 500 nm (“particle size stabilizing effective amount”). In embodiments where the clarity of the final article is important, the particle size is less than about 200 nm, in some embodiments less than about 100 nm, and in others still, less than about 50 nm. In one embodiment, at least about 20 weight % dispersing agent, is used in at least one solution to insure that the desired particle size is achieved. In some embodiments the molar ratio of dispersing agent unit to metal agent is at least about 1.5, at least about 2, and in some embodiments at least about 4. As used herein, dispersing agent unit is a repeating unit within the dispersing agent. In some embodiments it will be convenient to have the same concentration of dispersing agent in both solutions.

The upper concentration limit for dispersing agent in the solutions may be determined by solubility of the dispersing agent in the selected solvent, and ease of handling of the solutions. In one embodiment, each solution has a viscosity of less than about 50 cps. In one embodiment the product solution may have up to about 50 weight % dispersing agent. As described above, the metal agent and salt precursor solutions may have the same or different concentrations of dispersing agent. All weight % are based upon the total weight of all components in the solution.

In this embodiment, the concentration of metal agent and salt precursor in the respective metal agent and salt precursor solutions is desirably at least about at least about 1500 ppm up to the solubility limit for the metal agent or salt precursor in the selected solvent, in some embodiments between about 5000 ppm and the solubility limit, in some embodiments between about 5000 ppm and 50,000 ppm (5 wt %) and in other embodiments between about 5000 and about 20,000 ppm (2 wt %).

The mixing of the solutions may be conducted at room temperature, and may beneficially include stirring. Stirring speeds at or above which a vortex is created may be used. The selected stirring speed should not cause frothing, foaming or loss of solution from the mixing container. Stirring is continued throughout addition.

Mixing may be conducted at ambient pressure, or decreased pressure. In some embodiments, mixing may cause the solution to froth or foam. Foaming or frothing is undesirable as it may cause pockets of higher concentration of the metal salt to form, which results in larger than desired particle size. In these cases decreased pressure may be used. The pressure can be any pressure between ambient and the vapor pressure for the selected solvent. In one embodiment, where water is the solvent, the pressures may be between ambient and about 40 mbar.

The rate of addition of the salt precursor and metal agent solutions is selected to maintain a clear solution throughout mixing. Slight localized haze may be acceptable so long as the solution clears with stirring. Clarity of the solution may be observed visually or monitored using UV-VIS spectroscopy. Suitable addition rates may be determined by preparing a series of solutions having the desired concentration, and monitoring the clarity of the solution at different addition rates. This procedure is exemplified in Examples 26-31. Including dispersing agent in the salt precursor solution may also allow for faster rates of addition.

In another embodiment, where faster addition rates are desired, the metal agent and dispersing agent are allowed to mix under complex-forming conditions, including a complex-forming time before mixing with the salt precursor solution. It is believed that the dispersing agent in the metal agent solution forms a complex with the metal agent. In this embodiment, it is desirable to allow the metal agent to fully complex with the dispersing agent prior to combining the metal agent solution and the salt precursor solution. “Fully complexed” means that substantially all the metal ions have complexed with at least one dispersing agent. “Substantially all” means at least about 90%, and in some embodiments at least about 95% of said metal ions have complexed with at least one dispersing agent.

The complex-forming time may be monitored in solution via spectroscopy, such as via UV-VIS or FTIR. The spectra of the metal agent solution without the dispersing agent is measured. The spectra of the metal agent solution is monitored after addition of the dispersing agent, and the change in spectra is monitored. The complex-forming time is the time at which the spectral change plateaus.

Alternatively, complexation time may be measured empirically by forming a series of metal agent-dispersing agent solutions having the same concentration, allowing each solution to mix for a different time (for example 1, 3, 6, 12, 24, 72 hours and 1 week), and mixing each metal agent-dispersing agent solution batch-wise with the salt precursor solution. The metal agent-dispersing agent solutions which are mixed for complex-forming times will form clear solutions when the metal agent and salt precursor solutions are poured together directly without controlling the rate of addition. For example, 20 ml of metal agent solution may be added to 20 ml of salt precursor solution in 1 second or less.

Complexation conditions include complexation time (discussed above), temperature, ratio of the dispersing agent to the metal agent and stirring rates. Increasing the temperature, molar ratio of dispersing agent to metal agent and stirring rate, will decrease complexation time. Those of skill in the art will, with reference to the teachings herein, can vary the conditions to achieve the disclosed complexation levels.

If the metal agent and dispersing agent are not fully complexed, the mixing conditions may be selected to bias reactions in the mixture to forming the dispersing agent-metal agent complex over the formation of uncomplexed metal salt. This biasing may be achieved by controlling the (a) concentration of dispersing agent in the salt precursor, or the solution into which the salt precursor and metal agent solutions are added and (b) rate of mixing of the metal agent and salt precursor solutions.

Once the metal agent and salt precursor solutions have been mixed, the product solution may be dried. Any conventional drying equipment may be used such as freeze dryers, spray dryers and the like. Drying equipment and processes are well known in the art. An example of a suitable spray dryer is a cyclone spray dryer, such as those available from GEA Niro, Inc. For spray drying the temperature of the spray inlet is above the flash point for the selected solvent.

Freeze dryers are available from numerous manufacturers, including GEA Niro, Inc. Freeze drying temperatures and pressures are selected to sublimate the solvent as is well known by those of skill in the art. Any temperature within conventional ranges for the method selected may be used.

The product solution is dried until the resulting powder has a solvent content of less than about 10 weight %, in some embodiments less than about 5 weight % and in some embodiments less than about 2 weight %. Higher solvent concentrations may be appropriate where the solvent used to form the stabilized metal salt is compatible with the reaction mixture used to form the polymeric article. The powder comprises stabilized metal salt particles having a particle size of up to about 100 nm, up to about 50 nm, and in some embodiments up to about 15 nm as measured by transmission electron microscopy, photon correlation spectrometry or dynamic light scattering by dispersing in water.

The stabilized metal salt powder may be added directly to the reaction mixture. The amount of stabilized metal salt powder to be added may be readily calculated to provide the desired level of antimicrobial metal ion.

The reactive mixture, comprising the metal salt is reacted to form an antimicrobial polymeric article. The conditions for the reaction may be readily selected by those of skill in the art based upon the components in the reactive mixture. For example, where the antimicrobial polymeric article is a contact lens formed from free radical reactive components, the reactive mixture comprises an initiator and the reaction conditions may include curing with light or heat. Where antimicrobial metal salts which are photosensitive, such as AgI, AgCl and AgBr, exposure of the metal salt to wavelengths below the critical wavelength as described above converts the Ag⁺ to Ag⁰, which results in a darkening of the article in which the salt is incorporated. Accordingly, in one embodiment, when free radical reactive components are used, curing is conducted via exposure to visible light. In other embodiment, the reactive mixture further comprises at least one UV absorbing compound and is cured using visible light, heat or a combination thereof. In yet other embodiment, the reactive mixture further comprises at least one UV absorbing compound, a visible light photoinitiator and is cured using visible light.

The metal salts can be formed in or added to a variety of polymers. Suitable polymers may be selected based upon the intended use. For example, for food packaging applications polymers such as polyethylene terephthalate, high density polyethylene and polypropylene are commonly used for food and beverage containers and low density polyethylene is commonly used for plastic wraps.

Several implantable devices, such as joint replacements, are made using highly crosslinked ultra high molecular weight polyethylene (UHMWPE), which typically has a molecular weight of at least about 400,000, and in some embodiments from about 1,000,000 to about 10,000,000 as defined by a melt index (ASTM D-1238) of essentially 0 and reduced specific gravity of greater than 8 and in some embodiments between about 25 and 30.

Examples of suitable absorbable polymers suitable for use as yarns in making sutures and wound dressings include but are not limited to aliphatic polyesters which include but are not limited to homopolymers and copolymers of lactide (which includes lactic acid d-,l-and meso lactide), glycolide (including glycolic acid), Π-caprolactone, p-dioxanone (1,4-dioxan-2-one), trimethylene carbonate (1,3-dioxan-2-one), alkyl derivatives of trimethylene carbonate, δ-vaterolactone, β-butyrolactone, γ-butyrolactone, Π-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one and polymer blends thereof.

Sutures can also be made from non-absorbable polymer materials such as but are not limited to, polyamides (polyhexamethylene adipamide (nylon 66), polyhexamethylene sebacamide (nylon 610), polycapramide (nylon 6), polydodecanamide (nylon 12) and polyhexamethylene isophthalamide (nylon 61) copolymers and blends thereof), polyesters (e.g. polyethylene terephthalate, polybutyl terephthalate, copolymers and blends thereof), fluoropolymers (e.g. polytetrafluoroethylene and polyvinylidene fluoride) polyolefins (e.g. polypropylene including isotactic and syndiotactic polypropylene and blends thereof, as well as, blends composed predominately of isotactic or syndiotactic polypropylene blended with heterotactic polypropylene (such as are described in U.S. Pat. No. 4,557,264 issued Dec. 10, 1985 assigned to Ethicon, Inc. hereby incorporated by reference) and polyethylene (such as is described in U.S. Pat. No. 4,557,264 issued Dec. 10, 1985 assigned to Ethicon, Inc. and combinations thereof.

The body of the punctal plugs may be made of any suitable biocompatible polymer including, without limitation, silicone, silicone blends, silicone co-polymers, such as, for example, hydrophilic monomers of pHEMA (polyhydroxyethlymethacrylate), polyethylene glycol, polyvinylpyrrolidone, and glycerol, and silicone hydrogel polymers such as, for example, those described in U.S. Pat. Nos. 5,962,548, 6,020,445, 6,099,852, 6,367,929, and 6,822,016. Other suitable biocompatible materials include, for example: poly(ethylene glycol); poly(ethylene oxide); poly(propylene gycol); poly(vinyl alcohol); poly(hydroxyethyl methacrylate); poly(vinylpyrrolidone); polyacrylic acid; poly(ethyloxazoline); poly(dimethyl acrylamide); phospholipids, such as, for example, phosphoryl choline derivatives; polysulfobetains; polysaccharides and carbohydrates, such as, for example, hyaluronic acid, dextran, hydroxyethyl cellulose, hydroxyl propyl cellulose, gellan gum, guar gum, heparan sulfate, chondritin sulfate, heparin, and alginate; proteins such as, for example, gelatin, collagen, albumin, and ovalbumin; polyamino acids; fluorinated polymers, such as, for example, polytetrafluoroethylene (“PTFE”), polyvinylidene fluoride (“PVDF”), and teflon; polypropylene; polyethylene; nylon; and ethylene vinyl alcohol (“EVA”).

Polymeric parts of ultrasonic surgical instruments may be made from polyimides, fluora ethylene propene (FEP Teflon), PTFE Teflon, silicone rubber, EPDM rubber, any of which may be filled with materials such as Teflon or graphite or unfilled. Examples are disclosed in US20050192610 and U.S. Pat. No. 6,458,142.

Methods for the manufacture of the foregoing polymers are well known, and the stabilized metal salt particles may be readily incorporated via melt compounding or during polymerization. Suitable dispersing agents for each system may be readily selected by considering the thermal stability of the dispersing agent and the dispersing agent-metal agent complex.

In one embodiment the antimicrobial polymeric article is a lens. As used herein, the term “lens” refers to an ophthalmic device that resides in or on the eye. These devices can provide optical correction, therapeutic effect, cosmetic effect or a combination thereof. The term lens includes but is not limited to soft contact lenses, hard contact lenses, intraocular lenses, overlay lenses, ocular inserts, and optical inserts, such as, but not limited to punctal plugs.

Soft contact lenses may be made from silicone elastomers or hydrogels, which include but are not limited to silicone hydrogels, and fluorohydrogels. Preferably, the lenses of the invention are optically clear, with optical clarity comparable to lenses such as lenses made from etafilcon A.

Metal salts of the invention may be added to the soft contact lens formulations described in U.S. Pat. No. 5,710,302, WO 9421698, EP 406161, JP 2000016905, U.S. Pat. No. 5,998,498, U.S. patent application Ser. No. 09/532,943, U.S. Pat. No. 6,087,415, U.S. Pat. No. 5,760,100, U.S. Pat. No.5,776, 999, U.S. Pat. No. 5,789,461, U.S. Pat. No. 5,849,811, and U.S. Pat. No. 5,965,631. In addition, metal salts of the invention may be added to the formulations of commercial soft contact lenses. Examples of soft contact lenses formulations include but are not limited to the formulations of etafilcon A, genfilcon A, lenefilcon A, polymacon, acquafilcon A, balafilcon A, lotrafilcon A, lotrafilcon B, galyfilcon, senofilcon and comfilcon. In one embodiment the contact lens formulations are etafilcon A, balafilcon A, acquafilcon A, lotrafilcon A, lotrafilcon B, senofilcon, galyfilcon, comfilcon, in other embodiment, etafilcon A, galyfilcon, comfilcon and silicone hydrogels, as prepared in U.S. Pat. No. 5,998,498, U.S. patent application Ser. No. 09/532,943, a continuation-in-part of U.S. patent application Ser. No. 09/532,943, filed on Aug. 30, 2000, WO03/022321, U.S. Pat. No. 6,087,415, U.S. Pat. No. 5,760,100, U.S. Pat. No.5,776,999, U.S. Pat. No. 5,789,461, U.S. Pat. No. 5,849,811, and U.S. Pat. No. 5,965,631. These patents as well as all other patent disclosed in this paragraph are hereby incorporated by reference in their entirety. In one embodiment the metal salts of the present invention are added to lens materials which have a hydrophilicity index of at least about 41, as described in U.S. Ser. No. 11/757484. In one embodiment, the article is a contact lens formed from galyfilcon.

Hard contact lenses are made from polymers that include but are not limited to polymers of poly(methyl)methacrylate, silicon acrylates, silicone acrylates, fluoroacrylates, fluoroethers, polyacetylenes, and polyimides, where the preparation of representative examples may be found in U.S. Pat. No. 4,330,383. Intraocular lenses of the invention can be formed using known materials. For example, the lenses may be made from a rigid material including, without limitation, polymethyl methacrylate, polystyrene, polycarbonate, or the like, and combinations thereof. Additionally, flexible materials may be used including, without limitation, hydrogels, silicone materials, acrylic materials, fluorocarbon materials and the like, or combinations thereof. Typical intraocular lenses are described in WO 0026698, WO 0022460, WO 9929750, WO 9927978 and WO 0022459. U.S. Pat. Nos. 4,301,012; 4,872,876; 4,863,464; 4,725,277; 4,731,079. Metal salts may be added to hard contact lens formulations and intraocular lens formulations as described above.

Biomedical devices, including ophthalmic lenses may be coated to increase their compatibility with living tissue provided the coating does not prevent or undesirably reduce activity of antimicrobial metal salt. Therefore, the articles of the inventions may be coated with a number of agents that are used to coat lens. Alternatively, the stabilized metal salt particles may be conveniently added to any known coating compositions, and in one embodiment to solution compositions which are formed from solutions and reactive mixtures such as dip coating solutions, mold transfer coatings, reactive coatings and the like, following the teachings of the present invention. Suitable examples include, but are not limited to coatings using coupling agents or tie layers, such as those disclosed in U.S. Pat. Nos. 6,087,415 and US 200//0086160, latent hydrophilic coatings such as those disclosed in U.S. Pat. No. 5,779,943, polyethylene oxide star coatings such as those disclosed in U.S. Pat. No. 5,275,838, covalently bound coatings such as those disclosed in 4,973,493, coatings formed by the polymerization and crosslinking of reactive monomers contacted with the article to be coated as described in U.S. Pat. No. 5,135,297, graft polymerization coatings such as those disclosed in U.S. Pat. No. 6,200,626, non-reactive or complex forming coatings such as those disclosed in EP 1,287,060, U.S. Pat. No. 6,689,480 and WO2004/060431, “layer-by-layer coatings” such as those disclosed in EP 1252222, U.S. Pat. No. 7,022,379, U.S. Pat. No. 6,896,926, US 2004/0224098, US2005058844 and U.S. Pat. No. 6,827,966, mold transfer coatings, such as those disclosed in WO03/011551A1 and the surface modification processes disclosed in U.S. Pat. No. 5,760,100. Silicate coatings such as disclosed in U.S. Pat. No. 6,193,369, and plasma coatings such as disclosed in U.S. Pat. No. 6,213,604 may be applied over articles, such as ophthalmic devices which comprise antimicrobial metal salts. These applications and patents are hereby incorporated by reference for those procedures, compositions, and methods.

Many of the lens formulations cited above may allow a user to insert the lenses for a continuous period of time ranging from one day to thirty days. It is known that the longer a lens is in the eye greater the chance that bacteria and other microbes will build up on the surface of those lenses. The lenses of the present invention help prevent the build up of bacteria on a polymeric article, such as a contact lens.

Still yet further, the invention includes a method of reducing the adverse events associated with microbial colonization on a lens placed in the ocular regions of a mammal comprising, consisting of, or consisting essentially of, placing an antimicrobial lens comprising at least one antimicrobial metal salt on the eye of a mammal for at least about 14 days, and wherein said lens comprises at least about 0.5 μg of extractible antimicrobial metal after said at least 14 day period. In another embodiment the lens comprises at least about 0.5 μg of said extractible antimicrobial metal after at least 30 days. In this embodiment, the lenses may be worn continuously or may be worn in a daily wear mode (removed before sleeping and reinserted upon waking). Extraction of the antimicrobial metal salt may be determined using the conditions described above. In yet another embodiment, the lenses of the present invention comprising an initial concentration of antimicrobial metal salt sufficient to release 0.5 μg antimicrobial metal per day during the intended wear period. Intended wear period is the length of time a lens is recommended for wear by a patient.

The terms lens, antimicrobial lens, and metal salt all have their aforementioned meanings and preferred ranges. The phrase “adverse events associated with microbial colonization” include but are not limited to contact ocular inflammation, contact lens related peripheral ulcers, contact lens associated red eye, infiltrative keratitis, microbial keratitis, and the like. The term mammal means any warm blooded higher vertebrate, and the preferred mammal is a human.

The following test methods were used in the Examples.

Silver content of the lenses after lens autoclaving was determined by Instrumental Neutron Activation Analysis “INAA”. INAA is a qualitative and quantitative elemental analysis method based on the artificial induction of specific radionuclides by irradiation with neutrons in a nuclear reactor. Irradiation of the sample is followed by the quantitative measurement of the characteristic gamma rays emitted by the decaying radionuclides. The gamma rays detected at a particular energy are indicative of a particular radionuclide's presence, allowing for a high degree of specificity. Becker, D. A.; Greenberg, R. R.; Stone, S. F. J. Radioanal. Nucl. Chem. 1992, 160(1), 41-53; Becker, D. A.; Anderson, D. L.; Lindstrom, R. M.; Greenberg, R. R.; Garrity, K. M.; Mackey, E. A. J. Radioanal. Nucl. Chem. 1994, 179(1), 149-54. The INAA procedure used to quantify silver and iodide content in contact lens material uses the following two nuclear reactions:
1. In the activation reaction, ¹¹⁰Ag is produced from stable ¹⁰⁹Ag and ¹²⁸I is produced from stable ¹²⁷I after capture of a radioactive neutron produced in a nuclear reactor.
2. In the decay reaction, ¹¹⁰Ag (τ^{1/2}=24.6 seconds) and 128I (τ^{1/2}=25 minutes) decays primarily by negatron emission proportional to initial concentration with an energy characteristic to this radio-nuclide (657.8 KeV for Ag and 443 KeV for I).
   The gamma-ray emission specific to the decay of ¹¹⁰Ag and ¹²⁸I from irradiated. standards and samples are measured by gamma-ray spectroscopy, a well-established pulse-height analysis technique, yielding a measure of the concentration of the analyte.

Haze is measured by placing a hydrated test lens in borate buffered saline in a clear 20×40×10 mm glass cell at ambient temperature above a flat black background, illuminating from below with a fiber optic lamp (Titan Tool Supply Co. fiber optic light with 0.5″ diameter light guide set at a power setting of 4-5.4) at an angle 66° normal to the lens cell, and capturing an image of the lens from above, normal to the lens cell with a video camera (DVC 1300C:19130 RGB camera with Navitar TV Zoom 7000 zoom lens) placed 14 mm above the lens platform. The background scatter is subtracted from the scatter of the lens by subtracting an image of a blank cell using EPIX XCAP V 1.0 software. The subtracted scattered light image is quantitatively analyzed, by integrating over the central 10 mm of the lens, and then comparing to a −1.00 diopter CSI Thin Lens®, which is arbitrarily set at a haze value of 100, with no lens set as a haze value of 0. Five lenses are analyzed and the results are averaged to generate a haze value as a percentage of the standard CSI lens.

Subjective haze measurements were made using a Nikon SMZ1500 microscope in “dark field” mode, with the aperture set to full open. The lens to be evaluated was place in a glass Petri dish filled with SSPS and then put on the microscope inspection stage. The qualitative values from this method correspond roughly to the percent haze measured above as follows:

“High haze”: >˜100%

“Low haze”: <˜70

“Very low haze”: <˜40%

Color was measured as follows: samples were equilibrated in borate buffered sodium sulfate packing solution (SSPS) at room temperature. Excess moisture was removed from the lens surface. The lens was placed on a microscope slide and was rolled flat using a sponge swab. One drop of packing solution was place on the lens, and covered with a second microscope slide, insuring that there are no air bubbles on or under the lens. The lens is centered in front of a white background on the aperature of an X-Rite Model SP64 colorimeter, equipped with QA Master 2000 software. The instrument is calibrated using a 1⋅DAY ACUVUE contact lens. Three readings are taken, and the average is reported. Using the above described test, the L*a*b* values for a ⋅DAY ACUVUE contact lens measured 6 times and averaged are: L*=72.33±0.04, a*=1.39±0, b*=0.38±0.01.

The UV-Vis spectra of reactive mixtures was measured using a UVICAM UV300 instrument. The data was collected from 200-800 nm using one scan and a bandwidth of 1.5 nm. The baseline solvent used in listed in each of the Examples. The raw data was exported to Excel for plotting and analysis. The spectra were normalized over the wavelengths plotted for the purposes of comparison. For the monomers containing silver, the UV-Vis data was acquired 24 hours after addition of the silver-containing components.

UV-VIS spectra of lenses (%Transmission @ 200-800 nm) were acquired using an equilibrated Perkin Elmer Lambda 19 UV/VVIS scanning spectrometer (double monochormator system) across the range of 200-800 nm at an interval of 1 nm, with the following settings: 4 nm slit, 960 mn/min scan speed, smooth=2 nm, NIR sensitivity=3, lamp change=319.2 nm and detector change=860.8 nm. The lens is placed flat on a round sample holder and clip minimizing wrinkles and stretching. The lens and holder are placed in a cuvette filled with packing solution, and oriented such that the front curve faces the sample beam. The spectra is calculated using the software included on the instrument, using the equation: % Tₐᵥₑ=S/N, where S is the sum of % T at a specific region and N is the number of wavelength.

Distribution of metal salts throughout the plastic articles was measured using Electron Probe Microanalysis as described in Example 23.

Particle size was measured using laser light scattering or dynamic light scattering. For samples with a particle size range greater than about 500 nm a Horiba-LA930 laser diffraction particle size analyzer was used. The instrument check was performed from the blank % T values. One mL of the sample solution was introduced into the circulation bath which contained 150 mL of water as medium. A relative refractive index of 1.7-0.1 i and a circulation speed of 5 was used. The samples were ultrasonicated for two minutes prior to measurement using ultrasonication in the instrument. Triton® X-100 (commercially available from Union Carbide) (0.1%) was used as a surfactant in the analysis. Triplicate analysis was performed and the traces were compared to make sure that they coincided with each other. The instrument provided a report containing a graph of the particle size distribution along with values for the mean particle size.

For samples with a particle size range less than about 500 nm a Malvern 4700 dynamic light scattering apparatus was used. The instrument check was performed prior to analysis of the samples using NIST traceable standard size polystyrene particles. One ml of the sample was diluted to 20 ml with water and the samples were sonicated for one minute using Branson Ultrasonic probe and both relative refractive index and viscosity values were entered in the software. The instrument provides a report containing a graph of the particle size distribution along with values for the mean particle size.

Lenses were evaluated for efficacy against S. aureus using the following method. A culture of Staphylococcus aureus Clinical Isolate 031, was grown overnight in a tryptic soy medium (TSB). The culture was washed three (3) times in phosphate buffered saline (PBS, pH=7.4±0.2) and the bacterial pellet was resuspended in 10 mL of 2% TSB-PBS. The bacterial inoculum was prepared to result in a final concentration of approximately 1×10⁸ colony forming units/mL (cfu/mL). Serial dilutions were made in 2% TSB-PBS to achieve an inoculum concentration of 1×10⁴ cfu/mL.

The sterilized contact lenses were rinsed in three changes of 30 mL of phosphate buffered saline (PBS, pH=7.4+/±0.2) to remove residual solutions. Each rinsed contact lens was placed with 500 μL of the bacterial inoculum into separate test wells of a sterile tissue culture plate, which was then rotated in a shaker-incubator (100 rpm) for 20+/−2 hours at 35+/−2° C. Each lens and corresponding cell suspension was removed from the individual wells and placed in 9.5 mL of PBS containing 0.05% (w/v) Tween™ 80 (TPBS).

Lenses and corresponding cell suspension were then vortexed at 1600 rpm for 3 minutes, employing centrifugal force to disrupt adhesion of the remaining bacteria to the lens. The resulting supernatant was enumerated for viable bacteria using standard dilution and plate count techniques. The results of recovered viable bacteria associated with lenses were averaged.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

The following abbreviations were used in the examples
AHM=3-allyloxy-2-hydroxypropyl methacrylate
AMBN=2,2′-Azobis(2-Methylbutyronitrile)
BHT=butylated hydroxy toluene
Blue HEMA=the reaction product of reactive blue number 4 and HEMA, as described in Example 4 or U.S. Pat. No. 5,944,853
CGI 1850=1:1 (w/w) blend of 1-hydroxycyclohexyl phenyl ketone and bis (2,6-dimethyoxybenzoyl)-2,4-4-trimethylpentyl phosphine oxide
CGI 819=bis(2,4,6-trimethylbenzoyl)-phenylphosphineoxide
DI water=deionized water
DMA=N,N-dimethylacrylamide
DAROCUR 1173 2-hydroxy-2-methyl-1-phenyl-propan-1-one
EGDMA=ethyleneglycol dimethacrylate
HEMA=hydroxyethyl methacrylate
BAGE=boric acid ester of glycerin
IPA=Isopropyl alcohol
MAA=methacrylic acid
Macromer=silicone containing macromer as produced in Example 22
mPDMS=mono-methacryloxypropyl terminated polydimethylsiloxane (MW 800-1000)
Norbloc=2-(2′-hydroxy-5-methacrylyloxyethylphenyl)-2H-benzotriazole
HO-mPDMS=mono-(3-methacryloxy-2-hydroxypropyloxy)propyl terminated, mono-butyl terminated polydimethylsiloxane (MW 612), prepared as in Example 21
AgI Particles—AgI particles formed according to Synthetic Example 3
ppm=parts per million micrograms of sample per gram of dry lens
PAA=polyacrylic acid (Mw 2000)
PVP=polyvinylpyrrolidinone
PVA=polyvinyl alcohol
SiMMA=3-methacryloxy-2-hydroxypropyloxy)propylbis (trimethylsiloxy)methylsilane
SSPS=Borate Buffered Sodium Sulfate Packing Solution, made as described below
TAA=t-amyl alcohol
TBACB=tetrabutylammonium 3-chlorobenzoate
THF=tetrahydrofuran
TRIS=3-methacryloxypropyltris(trimethylsiloxy)silane
w/w=weight/total weight
w/v=weight/total volume
v/v=volume/total volume
The following compositions were prepared for use
   Tear-like fluid (TLF) buffer solution:

Tear-like fluid buffer solution (TLF Buffer) was prepared by adding the 0.137 g sodium bicarbonate (Sigma, S8875) and 0.01 g D-glucose (Sigma, G5400) to PBS containing calcium and magnesium (Sigma, D8662). The TLF buffer was stirred at room temperature until the components were completely dissolved (approximately 5 min).

A lipid stock solution was prepared by mixing the following lipids in TLF Buffer, with thorough stirring, for about 1 hour at about 60° C., until clear:

| | | |
|---|---|---|
| Cholesteryl linoleate (Sigma, C0289) | 24 | mg/mL |
| Linalyl acetate (Sigma, L2807) | 20 | mg/mL |
| Triolein (Sigma, 7140) | 16 | mg/mL |
| Oleic acid propyl ester (Sigma, O9625) | 12 | mg/mL |
| undecylenic acid (Sigma, U8502) | 3 | mg/mL |
| Cholesterol (Sigma, C8667) | 1.6 | mg/mL |

The lipid stock solution (0.1 mL) was mixed with 0.015 g mucin (mucins from Bovine submaxillary glands (Sigma, M3895,Type 1-S)). Three 1 mL portions of TLF Buffer were added to the lipid mucin mixture. The solution was stirred until all components were in solution (about 1 hour). TLF Buffer was added Q.S. to 100 mL and mixed thoroughly

The following components were added one at a time, and in the order listed, to the 100 mL of lipid-mucin mixture prepared above. Total addition time was about 1 hour.

| acid glycoprotein from Bovine plasma (Sigma, G3643) | 0.05 | mg/mL |
|---|---|---|
| Fetal Bovine serum (Sigma, F2442) | 0.1% | |
| Gamma Globulins from Bovine plasma (Sigma, G7516) | 0.3 | mg/mL |
| β lactoglobulin (bovine milk lipocaline) (Sigma, L3908) | 1.3 | mg/mL |
| Lysozyme from Chicken egg white (Sigma, L7651) | 2 | mg/mL |
| Lactoferrin from Bovine colostrums (Sigma, L4765) | 2 | mg/mL |

The resulting solution was allowed to stand overnight at 4° C. The pH was adjusted to 7.4 with 1N HCl. The solution was filtered and stored at -20° C. prior to use.

### Borate Buffered Sodium Sulfate Packing Solution (SSPS)

Packing solution contains the following ingredients in deionized H₂O:
0.18 weight % sodium borate [1330-43-4], Mallinckrodt
0.91 weight % boric acid [10043-35-3], Mallinckrodt
1.4 weight % sodium sulfate [7757-82-6], Sigma
0.005 weight % methylether cellulose [232-674-9] from Fisher Scientific

### Example 1

12.6 g of a 5% PVP (K12) solution in DI water was prepared. 3.94 g of 1% silver nitrate solution was added and mixed for 5 minutes using a magnetic stirring bar at room temperature. Then 3.47 g of a 1% sodium iodide solution was added and mixed for 5 minutes using a magnetic stirring bar at room temperature. A transparent silver iodide nanodispersion was obtained.

### Comparative Example 1

1.0 g of 1% AgNO₃ solution was added to 1.0 g of 1% NaI solution at room temperature. A very cloudy dispersion with AgI precipitation was obtained. Then 5 g of a 5% PVP (K12) solution was added with mixing using a magnetic stirrer for 48 hours. The precipitation did not clear up.

### Example 2

Example 1 was repeated except that the initial solution was made with PVA, 98% hydrolyzed, (Celvol 09-523, Celanese Chemicals, Dallas, Tex.) instead of PVP (K12). A transparent nanodispersion was obtained.

### Synthetic Example 1

A 5-liter glass reactor equipped with a stirrer, temperature control and a jacket for cooling and heating was charged with a mixture of the following compounds

| Component | Wt (gms) | |
|---|---|---|
| Ethanol | 2708.4 | g |
| HEMA | 291.95 | g |
| MAA | 5.96 | g |
| Norbloc | 2.92 | g |
| Blue HEMA | 0.0602 | g |
| TMPTMA | 0.30 | g |

The reactor temperature was raised to 71° C., and 2.11 g AMBN was added. The AMBN dissolved and the reactor was blanketed with a slow stream of nitrogen. The temperature was held at 71° C. for 20 hours.

Five 1-liter jars with screw lids and equipped with magnetic stirring bars were prepared and the crude product was poured into the jars, 600 g in each. The solution was heated to 60° C. in a water bath while constantly stirring with a magnet stirrer. Then, 54 g heptane (9%) was added and the solution re-heated to 60° C. The stirring was stopped and the jars placed in a water bath at 60° C. The temperature was ramped down to 24° C. over 20 hours. The top phase was now a clear fluid and the bottom phase was semi solid. The top phase was the biggest (about 80% of total jar) but with low polymer solids content (around 1.5-2.5%).

The top phases in each jar were discarded and the bottom phases were redissolved in aqueous ethanol in order to obtain 2125 g of polymer solution characterized by the following: 12% solids and 3% water.

This solution was spray dried using a Mini Spray Dryer B-290, equipped with an Inert Loop, an Outlet Filter, and a High Performance Cyclone at the following parameters:

| Inlet temp. | Inert Loop | Outlet temp. | Spray flow | Aspirator | Pump |
|---|---|---|---|---|---|
| 120° C. | −20° C. | 50° C. | 30 mm | 80% | 65% |

This resulted in 250 g of a fine white, fluffy powder that was about 97% dry. The powder was transferred to a number of 1 liter flasks (about 77 grams in each) equipped with a magnetic stirring bar. The flasks were vacuum treated overnight at 100-130° C. at vacuum pressure of less than 30 mbar to further dry out the material.

The next morning, the vacuum was broken with a dry argon atmosphere, and the flasks were transferred to a box with a controlled dry nitrogen atmosphere. The gross weight of the flasks was determined after cooling off. In each 1 liter flask, 300 g NMP (N-methylpyrrolidone anhydrous; purum; absolute; over molecular sieves, from Fluka) was added to fully dissolve the powder and the flasks were checked for homogeneity. MAH (methacrylic anhydride 98% pure) was weighed in a 50cc cylindrical glass container and 50 g NMP was added to dilute the MAH before transfer. Another 50 g NMP was used to flush the glass container, to ensure full transfer. Triethylamine (puriss p.a. from Fluka) was added directly using a finn pipette. Lids were tightened and sealed with tape and nitrogen flow was turned off. The reaction was allowed to run for about 40 hours.

The polymer produced above was purified as follows. 75 g polymer was dissolved in 400 mL NMP. Two 5-liter glass beakers were charged each with 4 liters of DI water, 30 mL fuming HCl (hydrochloric acid) and a magnetic stirring bar. The functionalized product from the previous reaction was poured gradually into the beakers, 200 mL in each at a rate of about 10 mL/sec. A precipitation occurred and the aqueous phase was removed. The remaining swollen polymer was redissolved in 300 mL ethanol.

Two more 5-liter glass beakers were charged each with 4 liters of DI water and a magnetic stirring bar. The polymer/ethanol solution was poured into the two 5-liter glass beakers filled with 2×4 L DI water and a precipitation occurred again. The aqueous phase was removed and fresh DI water was added in order to further extract residual HCl. After about 12 hours the aqueous phase was removed and the weight of the swollen polymeric material was determined (about 120 grams).

The swollen polymeric material was re-dissolved in ethanol to obtain a % solids content of 13±0.5%, subsequently the solution was filtered trough a 25 mm GD/X 0.45 μm Whatmann filter. The solution was spray dried using a Mini Spray Dryer B-290, equipped with an Inert Loop, an Outlet Filter, and a High Performance Cyclone. The following parameters were applied:

| Inlet temp. | Inert Loop | Outlet temp. | Spray flow | Aspirator | Pump |
|---|---|---|---|---|---|
| 79° C. | −20° C. | 43° C. | 30 mm | 80% | 26% |

This resulted in about 155 g of a fine white, fluffy powder.

### Example 3

The copolymer made in the Synthetic Example 1 (3.49 g) was mixed with 4.9 g of a master batch solution (containing 99.89% propylene glycol as diluent, 1.10% Dimethoxybenzoyl bis(acyl) phosphine oxide as a photoinitiator, and 0.011% 4-Methoxyphenol as an inhibitor. 2 g the nanodispersion prepared in Example 1 was weighed and mixed with the copolymer/master batch solution. The resulting mixture was centrifuged at 2500 rpm for 15 minutes to remove trapped air. A transparent prepolymer was obtained.

The prepolymer was dispensed into thermoplastic contact lens molds (front and backcurves made from polystyrene) that had been degassed under nitrogen for 12 hours. The prepolymer was irradiated in the molds at 30 mW/cm² light intensity at room temperature for 30 seconds in air at 20° C. The lenses were then hydrated in DI water at 20° C., for 20 minutes, packaged in borate buffered sodium sulfate packaging solution (SSPS) and sterilized at 121° C. for 18 minutes. The lenses have very low haze as measured under a darkfield microscope. The average silver content for five lenses measured using Neutron Activation technique, was found to be 9.72 micrograms with a standard deviation of 0.16 micrograms/lens.

### Example 4

0.339 g PVP (K12) powder was added to 3.487 g of a 1% NaI solution and mixed for 10 minutes to form salt precursor solution A. PVP (K12-0.266 g), was slowly added into 4.29 g of a 1% solution of AgNO₃ to form metal agent solution B. The salt precursor solution A (0.379 g), was added to 17.603 g of the monomer mixture shown in Table 1, below and mixed for 3 minutes. Metal agent solution B (0.3963 g) was then added to the monomer mixture and stirred for 10 minutes.

The monomer mixture was degassed under vacuum (29″ Hg) for 20 minutes. The monomer mixture was dispensed into thermoplastic contact lens molds (front and backcurves made from polystyrene) and irradiated at 5 mW/cm² light intensity at room temperature for 6 minutes under nitrogen. The lenses were then hydrated in DI water at 20° C., packaged in SSPS and sterilized in an autoclave at 121° C. for about 20 minutes. The lenses have very low haze as measured under a darkfield microscope. The silver content was measured using Neutron Activation technique and was 4.7 micrograms with a standard deviation of 0.11 micrograms/lens.

TABLE 1

| Component | Parts by wt. |
|---|---|
| HEMA | 58.08 |
| MAA | 0.96 |
| Blue HEMA | 0.07 |
| EGDMA | 0.71 |
| Darocur 1173 | 0.14 |
| BAGE | 40 |

### Example 5

PVP (K12, 0.946 g) was slowly added to 30.7 g of the reactive mixture listed in Table 1 and dissolved by mixing for 25 minutes. 0.0177 g AgNO₃ (solid) was added and mixed until dissolved. Then 0.0300 g NaI (solid) was added and the mixture was mixed at room temperature for 1 hour to form a particle containing reactive mixture. The particle containing reactive mixture was degassed under vacuum (29″ Hg) for 10 min. The particle containing reactive mixture was dispensed in contact lens molds (front and backcurves made from polystyrene), cured, hydrated, packaged sterilized as described in Example 4. The lenses had very low haze as measured under a darkfield microscope. The silver content was measured using Neutron Activation technique and was 12.8 micrograms with a standard deviation of 0.4 micrograms/lens.

### Examples 6-13

In each of the following examples two mixtures were made. A salt precursor mixture (“SPM”), was made by mixing the reactive mixture listed in Table 1, PVP (K12) and NaI in the amounts listed in Table 2. The concentration of PVP (wt %) is listed as the weight % PVP in the particle containing reactive mixture. Metal agent mixture (“MAM”) was made by mixing the reactive mixture listed in Table 1 and AgNO₃ in the amounts listed in Table 2. Each mixture was mixed until all components were incorporated and a transparent mixture was formed (about 5 to about 19 hours). In each example, an approximately equal volume of salt precursor mixture, SPM and metal agent mixture, MAM were mixed to form a reactive mixture having the molar ratios of NaI to AgNO₃, listed in column 2 of Table 3. Each reactive mixture was mixed for ≥30 minutes, except for Example 8, which was mixed for 1 hour. The reactive mixtures were degassed under the conditions listed in Table 3. Each of the degassed reactive mixtures was dispensed, cured, hydrated, packaged sterilized as described in Example 4. The haze of the lenses was measured under a darkfield microscope. The silver content was measured using Neutron Activation technique. The targeted silver uptake in all lenses was about 10 μg. The results are reported in Table 3, below.

TABLE 2

| | | | gm | Gm | [PVP] |
|---|---|---|---|---|---|
| | gm Nal | gm RMM | AgNO₃ | RMM in | (k12) |
| Ex # | in SPM | in SPM | in MAM | MAM | (wt %) |
| 6 | 0.11 | 29.6 | 0.067 | 35 | 0.5 |
| 7 | 0.051 | 40 | 0.42 | 40 | 1 |
| 8 | 0.017 | 20 | 0.02 | 20 | 1.6 |
| 9 | 0.017 | 20 | 0.03 | 20 | 0.5 |
| 10 | 0.019 | 20 | 0.04 | 20 | 0.6 |
| 11 | 0.73 | 261 | 0.2 | 269 | 0.1 |
| 12 | 0.039 | 40 | 0.04 | 40 | 2.6 |
| 13 | 0.039 | 40 | 0.04 | 40 | 2.6 |

TABLE 3

| | | [PVP] | | Rip- | Haze | Haze | |
|---|---|---|---|---|---|---|---|
| Ex | NaI:AgNO₃ | (k12) | | ening | pre | post | Lens |
| # | molar | (wt %) | Dt | Cond. | Sterilizn | Sterilizn | Color |
| 6 | 1.9 | 0.5 | 5 | N/A | Very low | Low | Normal |
| 7 | 1.4 | 1 | 5 | N/A | Very low | Low | Normal |
| 8 | 0.98 | 1.6 | 5 | N/A | Very low | Low | Yellow |
| 9 | 0.71 | 0.5 | 5 | N/A | Very low | Low | Light |
| | | | | | | | Brown |
| 10 | 0.58 | 0.6 | 5 | N/A | Very low | Low | Brown |
| 11 | 4.1 | 0.1 | 5 | N/A | Very low | Very | Normal |
| | | | | | | low | |
| 12 | 1.1 | 2.6 | 50 | N/A | Very low | Very | Normal |
| | | | | | | high | |
| 13 | 1.1 | 2.6 | 50 | 70 C. | Very low | Low | |
| | | | | for | | | |
| | | | | 20 min | | | |

Dt = degas time in minutes

Examples 6 and 7, which contained a molar excess of NaI, had very low haze prior to sterilization, low haze after sterilization, and normal color. In comparison, Examples 8, 9 and 10, which were made using the same conditions, but an excess of AgNO₃, displayed a yellow, light brown and brown color respectively. Thus, Examples 6 and 7 show that process conditions which insure conversion of the metal agent to metal salt provide articles having improved color, particularly when the metal agent is more photosensitive than the metal salt.

Example 12, which had 2.6% PVP and a degas step of 50 minutes, displayed very low haze prior to sterilization, but high haze after sterilization, suggesting that particle ripening may have taken place during sterilization. However, when a particle ripening step was added before the reactive mixture was cured (Example 13, 70° C. for 20 minutes) the resulting lenses displayed low haze after sterilization.

### Example 14

The reactive mixture in Table 4 below was prepared. The reactive components are reported as weight percent of all reactive components (excluding diluent) and the diluent is weight percent of final reaction mixture. AgNO₃ solid (0.040 g) was added to 28.09 g of the monomer mixture. Then NaI (solid, 0.0427 g) was added to the mixture and mixed at room temperature for 1 hour. After mixing, there were still solids at the bottom of the container. The reactive mixture was dispensed into thermoplastic contact lens molds (front and backcurves made from Zeonor® obtained from Zeon, Corp.) and irradiated at 5 mW/cm² light intensity at room temperature for 10 minutes under N₂. The lenses were then hydrated in DI water at 25° C., packaged in borate buffered sodium sulfate packaging solution and sterilized in an autoclave at 121° C. for about 20 minutes. The lenses had very low haze as measured under a darkfield microscope, but had a slight black tint. The silver content was 6.2 micrograms with a standard deviation of 0.21 micrograms/lens, using Neutron Activation technique.

TABLE 4

| Component | |
|---|---|
| SiMMA | 30 |
| PVP (K90) | 6 |
| DMA | 31 |
| MPDMS | 23 |
| HEMA | 7.5 |
| Norbloc | 1.5 |
| CGI 819 | 0.23 |
| EGDMA | 0.75 |
| Blue HEMA | 0.02 |
| PVP (MW 2,500) | 11 |
| TAA | 29 |

### Example 15

PVP K12 (9.29 g) was slowly added in 200.00 g TPME while stirring, and mixed for 20 minutes. Then 0.7040 g silver nitrate solid was added into the solution to form a metal agent solution. The metal agent solution was stirred using a magnetic stirrer for 6 hours.

Sodium iodide (0.8880 g) was added into 200.13 g TPME to form a salt precursor solution. The salt precursor solution was stirred using a magnetic stirrer for 6 hours. The metal agent solution (170.89 g) was mixed into the salt precursor solution (171.21 g) with constant stirring. A transparent nanodispersion was obtained. The solution was mixed for 25 minutes. The total nanodispersion was then mixed into 500.20 g of reactive mixture listed in Table 5 below:

TABLE 5

| Component | Parts by Weight |
|---|---|
| SiMMA | 30.00 |
| mPDMS1000 | 22.00 |
| DMA | 31.00 |
| HEMA | 8.50 |
| EGDMA | 0.75 |
| PVP K90 | 6.00 |
| Norbloc | 1.50 |
| Blue HEMA | 0.02 |
| CGI 819 | 0.23 |

The reactive mixture was degassed at −29″ (740 mm) Hg for 15 minutes. The reactive mixture was dispensed into thermoplastic contact lens molds (front and backcurves made from Zeonor® obtained from Zeon, Corp.) and irradiated at 5 mW/cm² light intensity at room temperature for 6 minutes in nitrogen. The lenses were then hydrated in DI water at 20° C. for 30 minutes, then in 70% IPA for 60 minutes, then rinsed in DI water for 1 minutes and then staged in DI water for >2 hours, all at room temperature. The lenses were then inspected, packaged in SSPS and sterilized in an autoclave at 121° C. for 18 minutes.

The lenses had and average silver uptake of 10.70 ug with a standard deviation of 0.2 ug (of 5 lenses). The haze of the lenses was 68% with a standard deviation of 8.9% (of 5 lenses).

### Example 16

419.5 g of a reactive mixture was made from the components listed in Table 6.

HEMA was added to TPME to form a HEMA/TPME (HEM:TPME 5.1:10) solution and mixed for 1 hour in a clean amber bottle.

A metal agent mixture was formed by slowly adding 7 g PVP (K12) to 70.0 g of the HEMA/TPME solution in a clean amber bottle and stirring with a magnetic stirring bar. The metal agent mixture was mixed until all PVP (K12) had been dissolved. AgNO₃ (0.49 g) was added and mixed for 6 hours until all solid was dissolved.

A salt precursor mixture was formed by adding 0.42 g NaI to 30 g of the HEMA/TPME solution in an amber bottle and mixing with magnetic stir bar for 6 hours, until all solid were dissolved.

The metal agent (67.02 g) mixture was slowly poured into the salt precursor mixture while stirring, and mixed for 1 hour. A transparent dispersion containing the metal salt AgI was obtained.

A reactive mixture having the components listed in Table 6 was prepared. The reactive components (419.5 g) and the metal salt dispersion (80.5 g) were mixed in an amber bottle and mixed for greater than about 24 hours. The reactive mixture was then filtered through 3 μg filter, and degassed under −29″ Hg for 15 minutes.

TABLE 6

| Components | Parts by Weight |
|---|---|
| SiMMA | 18 |
| MPDMS1000 | 13.2 |
| DMA | 18.6 |
| t-amyl alcohol | 29 |
| EGDMA | 0.45 |
| Norbloc | 0.9 |
| Blue HEMA | 0.012 |
| CGI 819 | 0.138 |
| PVP K90 | 3.6 |

The reactive mixture was dispensed into thermoplastic contact lens molds (front and backcurves made from Zeonor® obtained from Zeon, Corp.) and irradiated at 5 mW/cm² light intensity at room temperature for 6 minutes in nitrogen. The lenses were then hydrated in DI water at 20° C. for 30 minutes, then in 70% IPA for 60 minutes, then rinsed in DI water for 1 minutes and then staged in DI water for >2 hours, all at room temperature. The lenses were then inspected, packaged in borate buffered sodium sulfate packaging solution and sterilized in an autoclave at 121° C. for 18 minutes.

The lenses had and average silver uptake of 10.60 ug with a standard deviation of 0.2 ug (of 5 lenses). The haze of the lenses was 38.6% with a standard deviation of 4.3% (of 5 lenses).

### Example 17

0.0243 g PVP K12 was slowly added into 10.0037 g TPME, and mixed for 20 minutes using a magnetic stirrer. 0.0199 g of silver nitrate is then added into the solution, and the solution was mixed for 4 hours at room temperature to obtain solution A. 0.054 g sodium iodide solid was added into 10.0326 g TPME, and mixed for 4 hours at room temperature to obtain solution B. Solution A was poured into solution B, and mixed for 20 minutes to obtain a transparent nanodispersion of silver iodide in TPME.

4.20 g of the silver iodide nanodispersion prepared above was added into 5.13 g of monomer mixture that has the composition as shown in Table 7 below, and mixed for 12 hours. The monomer was then degassed at 22″ Hg vacuum for 20 minutes. The reactive mixture was dispensed into thermoplastic contact lens molds (front and backcurves made from Zeonor® obtained from Zeon, Corp.) and irradiated at 5 mW/cm² light intensity at room temperature for 6 minutes in nitrogen. The lenses were then hydrated in DI water at 20° C. for 30 minutes, then in 70% IPA for 60 minutes, then rinsed in DI water for 1 minutes and then staged in DI water for >2 hours, all at room temperature. The lenses were then inspected, packaged in borate buffered sodium sulfate packaging solution and sterilized in an autoclave at 121° C. for 18 minutes.

The lenses have silver content of 12 ug with a standard deviation of 0.1 ug (of 5 samples). The haze was 84% with a standard deviation of 4 (of 5 samples).

TABLE 7

| Component | Parts by wt. |
|---|---|
| HO-mPDMS | 55% |
| DMA | 19.53% |
| HEMA | 8% |
| TEGDMA | 3% |
| Norbloc | 2.2% |
| PVP K90 | 12% |
| Blue HEMA | 0.02% |
| CGI 819 | 0.25% |

### Comparative Example 2

Cured and hydrated galyfilcon lenses, available from Vistakon as ACUVUE ADVANCE® brand contact lenses, were placed in deionized water in a blister pack. The excess deionized water was removed and 0.8 mL salt precursor mixture (1100 ppm NaI in DI) was added to the blister containing the lens and left overnight at room temperature. The salt precursor mixture was removed and 0.8 mL of metal agent mixture (700 ppm silver nitrate and 5% PVP (k90) in DI) was added. After 3 minutes the metal salt mixture was removed and deionized water (900 μL) was added to the blister, left for approximately five minutes, and finally removed. The deionized water treatment was repeated two more times and the lenses were transferred to glass vials containing SSPS. The vials were sealed and autoclaved at 122° C. for 30 minutes. The lenses were analyzed by INAA, and contained about 16 μg Ag.

### Example 18 and Comparative Example 2

The relative silver content of the lenses of Example 6 and Comparative Example 2 were measured using EPM to determine the silver content distribution throughout the contact lens.

The samples were prepared for profile analyses by mounting the whole lens vertically in a 25 mm diameter aluminum holder that had been cut in half and drilled and tapped for two machine screws to clamp the specimen. The lens was clamped so that half of the material was above the surface of the holder. A clean single edge razor was then used to slice the lens in half in one smooth stroke to avoid tearing the cut surface. These samples were then carbon coated in a vacuum evaporator to ensure conductivity. The far edge of these samples was dabbed with colloidal carbon paint for better conductivity.

A strip from near the diameter of the remaining half of the lens was sliced from remaining the lens half and was carefully placed on a 25 mm diameter holder, with two double sided carbon “sticky tabs” on the top surface, with the concave surface up.

The convex lens surface was analyzed by mounting the remaining chord of lens material convex side up on two “sticky tabs”. A sheet of clean Teflon material (0.032″ thick) was used to press the contact lens flat to the carbon “sticky tabs”. These samples were coated with 20-40 nm of Spec-Pure graphite in a carbon vacuum evaporator. The far edge of these samples was daubed with colloidal carbon paint for better conductivity.

The samples were analyzed using either Cameca SX-50 (1988) or SX-100 (2005) automated electron microprobe with 4 wavelength spectrometers, using analytical conditions of 20 keV, 50 nA and a 20 μm defocused beam size for analyzing the surface of the lens. The beam size was reduced to 5 microns for the analysis of profiles. Counting time was 160 seconds on peak and 80 seconds on each off-peak.

Background positions were selected to be free from spectral interferences. Background intensities were calculated by linear interpolation between the off-peak positions. Intensities were also corrected for detector dead time, beam drift and standard intensity drift. No significant drift was noted for any analyses. The detection limit was about 40 ppm for Ag.

The acquisition of profile analyses was done by locating the convex side (front curve) of the profile surface and starting all traverses from that point. Surface analyses were performed by starting at one side of the strip of lens material and using 250 or 500 um steps across the entire lens. This was generally on the order of 8-12 mm in total distance (25 to 50 data points per sample surface). All data points were manually confirmed for Z focus to be sure that spectrometer defocusing did not occur for samples that were not perfectly flat after waiting for approximately 4 hours for the sample surface to stabilize with respect to Z focus.

Ag metal was used as the primary standard for Ag. Standards and unknowns were coated with 20 nm of Spec-Pure graphite and run under the conditions described above, except that the counting time for the standards was 10 seconds on-peak and 5 seconds on each off-peak.

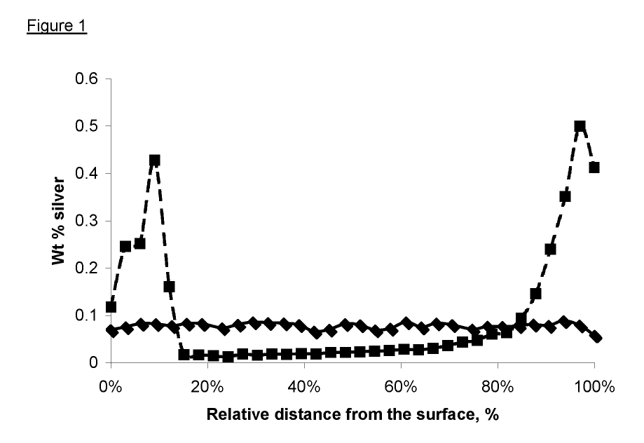

FIG. 1 is a compilation graph of silver distribution through the lenses of Example 16 and Comparative Example 1, where the silver is precipitated in the lens after lens formation. As can be seen from FIG. 1, the concentration of metal salt in the lenses of Example 21 is consistent throughout the entire lens (shown by the line connecting the squares). For the lenses of Comparative Example 2, FIG. 1 also shows that the lenses analyzed had high concentrations of silver within 20% of the front and back surfaces of the lens, but very little silver in the center (as shown by the line connected with diamonds).

### Example 19

Lenses made according to Example 16 were evaluated for silver release from the lens using the following method.

The lenses to be tested were blotted using sterile guaze to remove excess liquid and then transferred, 1 lens/well, into sterile 24 well cell culture plates containing 1 ml TLF in each well. The plates were covered to prevent evaporation and dehydration and incubated at 35° C. with agitation of at least 100 rpm. Every 24 hours the lenses were transferred into fresh 1 ml volumes of TLF. At each time interval where measurements were made, a minimum of 3 lenses were removed from their wells, and rinsed 3-5 times with 100 mls of PBS. The lenses were blotted on paper towels to remove excess liquid and transferred to propylene scintillation vials (one lens/vial). The silver content was analyzed by Neutron Activation Analysis.

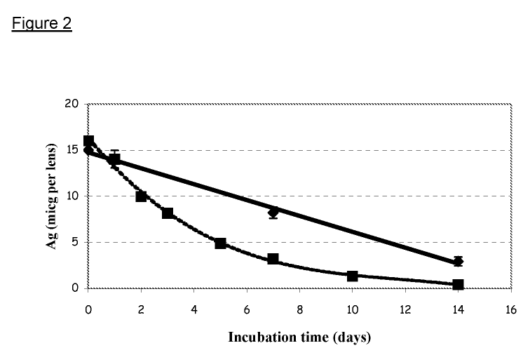

The lenses of Comparative Example 2 were also tested as described above. The results for both lenses are shown in Table 3. In FIG. 2, the solid line connecting the diamond shaped points are the results from the lenses of Example 16 and the dotted line connecting the square points are the results from the evaluation of the lenses of Comparative Example 2. FIG. 2 clearly shows that the lenses of the present invention release the antimicrobial metal more slowly and consistently than the lenses of Comparative Example 2 (where the silver salt was precipitated in the lens after the lens was formed).

### Example 20

The lenses of Example 16 and Comparative Example 2 were evaluated for efficacy against bacteria using the following method. A culture of Pseudomonas aeruginosa, ATCC #15442 (American Type Culture Collection, Rockville, Md.), was grown overnight in a tryptic soy medium. The culture was washed three (3) times in phosphate buffere saline (PBS, pH=7.4±0.2) and the bacterial pellet was resuspended in 10 mL of 2% TSB-PBS. The bacterial inoculum was prepared to result in a final concentration of approximately 1×10⁸ colony forming units/mL (cfu/mL). Serial dilutions were made in 2% TSB-PBS to achieve an inoculum concentration of 1×104 cfu/mL.

The sterilized contact lenses were rinsed in three changes of 30 mL of phosphate buffered saline (PBS, pH=7.4+/−0.2) to remove residual solutions. Each rinsed contact lens was placed with 500 μL of the bacterial inoculum into a separate test well of a sterile tissue culture plate, which was then rotated in a shaker-incubator (100 rpm) for about 20 hours at 35+/−2° C. Each lens was removed from the glass vial, rinsed five (5) times in three (3) changes of PBS to remove loosely bound cells. After incubation, three lenses of each lens type were removed to measure the initial bacterial efficacy (described below) and the remaining lenses were transferred into the wells of new microtiter plates containing 500 μL TLF as described above.

The remaining lenses were incubated for 7 and 14 days in individual tissue culture wells with 1 ml/lens of TLF with the lenses transferred to fresh TLF solution every 24 hours.

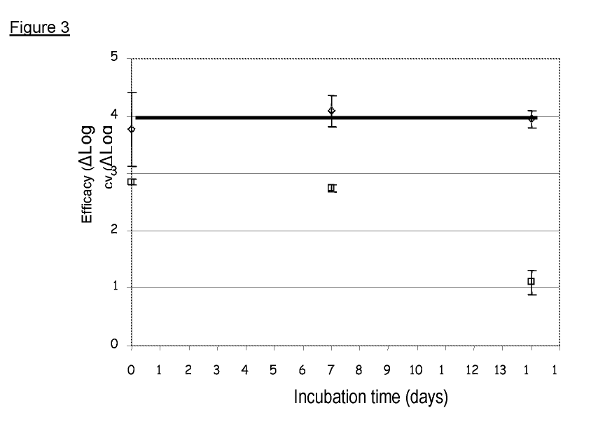

At the end of the incubation period (post-incubation, 7 and 14 days) the lenses to be measured were removed from their wells rinsed five (5) times with 3 changes of PBS to remove loosely bound cells, placed into about 10 mL of PBS containing 0.05% (w/v) Tween™ 80, and vortexed at 2000 rpm for 3 minutes, employing centrifugal force to disrupt adhesion of the remaining bacteria to the lens. The resulting supernatant was enumerated for viable bacteria using an RBD 3000 flow cytometer and the results of detectable viable bacteria attached to 3 lenses were averaged. The results are presented in FIG. 3. ACUVUE® ADVANCE™ with Hydraclear™ brand contact lenses, available from Vistakon, were used as a control.

The results for the lenses of Example 16 and Comparative Example 2 are shown in FIG. 3. The solid line connecting the diamond shaped points are the results from the lenses of Example 16 and the dotted line connecting the square points are the results from the evaluation of the lenses of Comparative Example 2. FIG. 3 shows that the lenses of the present invention display a consistent 4 log reduction of bacteria (Pseudomonas aeruginosa) over 14 days. Unlike the lenses of the present invention, the lenses of Comparative Example 2 displayed a 3 log reduction for the first 7 days, which then decrease over the remaining period of evaluation to about a 1 log reduction at 14 days. Accordingly, the lenses of the present invention displayed efficiacy, which was both greater and longer lasting than the lenses of Comparative Example 2.

### Example 21

To a stirred solution of 45.5 kg of 3-allyloxy-2-hydroxypropyl methacrylate (AHM) and 3.4 g of butylated hydroxy toluene (BHT) was added 10 ml of Pt (0) divinyltetramethyldisiloxane solution in xylenes (2.25% Pt concentration) followed by addition of 44.9 kg of n-butylpolydimethylsilane. The reaction exotherm was controlled to maintain reaction temperature of about 20° C. After complete consumption of n-butylpolydimethylsilane, the Pt catalyst was deactivated by addition of 6.9 g of diethylethylenediamine. The crude reaction mixture was extracted several times with 181kg of ethylene glycol until residual AHM content of the raffinate was <0.1%. 10 g of BHT was added to the resulting raffinate, stirred until dissolution, followed by removal of residual ethylene glycol affording 64.5 kg of the OH-mPDMS. 6.45 g of 4-Methoxy phenol (MeHQ) was added to the resulting liquid, stirred, and filtered yielding 64.39 kg of final OH-mPDMS as colorless oil.

### Synthetic Example 2

### Macromer Preparation

To a dry container housed in a dry box under nitrogen at ambient temperature was added 30.0 g (0.277 mol) of bis(dimethylamino)methylsilane, a solution of 13.75 mL of a 1M solution of TBACB (386.0 g TBACB in 1000 mL dry THF), 61.39 g (0.578 mol) of p-xylene, 154.28 g (1.541 mol) methyl methacrylate (1.4 equivalents relative to initiator), 1892.13 (9.352 mol) 2-(trimethylsiloxy)ethyl methacrylate (8.5 equivalents relative to initiator) and 4399.78 g (61.01 mol) of THF. To a dry, three-necked, round-bottomed flask equipped with a thermocouple and condenser, all connected to a nitrogen source, was charged the above mixture prepared in the dry box.

The reaction mixture was cooled to 15° C. while stirring and purging with nitrogen. After the solution reached 15° C., 191.75 g (1.100 mol) of 1-trimethylsiloxy-1-methoxy-2-methylpropene (1 equivalent) was injected into the reaction vessel. The reaction was allowed to exotherm to approximately 62° C. and then 30 mL of a 0.40 M solution of 154.4 g TBACB in 11 mL of dry THF was metered in throughout the remainder of the reaction. After the temperature of reaction reached 30° C. and the metering began, a solution of 467.56 g (2.311 mol) 2-(trimethylsiloxy)ethyl methacrylate (2.1 equivalents relative to the initiator), 3812 g (3.63 mol) n-butyl monomethacryloxypropyl-polydimethylsiloxane (3.3 equivalents relative to the initiator), 3673.84 g (8.689 mol), TRIS (7.9 equivalents relative to the initiator) and 20.0 g bis(dimethylamino)methylsilane was added.

The mixture was allowed to exotherm to approximately 38-42° C. and then allowed to cool to 30° C. At that time, a solution of 10.0 g (0.076 mol) bis(dimethylamino)methylsilane, 154.26 g (1.541 mol) methyl methacrylate (1.4 equivalents relative to the initiator) and 1892.13 g (9.352 mol) 2-trimethylsiloxy)ethyl methacrylate (8.5 equivalents relative to the initiator) was added and the mixture again allowed to exotherm to approximately 40° C. The reaction temperature dropped to approximately 30° C. and 2 gallons of THF were added to decrease the viscosity. A solution of 439.69 g water, 740.6 g methanol and 8.8 g (0.068 mol) dichloroacetic acid was added and the mixture refluxed for 4.5 hours to de-block the protecting groups on the HEMA. Volatiles were then removed and toluene added to aid in removal of the water until a vapor temperature of 110° C. was reached.

The reaction flask was maintained at approximately 110° C. and a solution of 443 g (2.201 mol) TMI and bismuth K-KAT 348 (5.94 g) were added. The mixture was reacted until the isocyanate peak was gone by IR. The toluene was evaporated under reduced pressure to yield an off-white, anhydrous, waxy reactive monomer. The macromer was placed into acetone at a weight basis of approximately 2:1 acetone to macromer. After 24 hrs, water was added to precipitate out the macromer and the macromer was filtered and dried using a vacuum oven between 45 and 60° C. for 20-30 hrs.

### Synthetic Example 3

### Formation of AgI Nanodispersion

Metal agent and salt precursor solutions were formed as follows: 10,000 ppm AgNO₃ was dissolved with stirring in 200 gm of a 50 w/w % solution of PVP K12 in DI water. NaI (10,000 ppm) was dissolved with stirring in 200 gm of a 50 w/w % solution of PVP K12 in DI water. The metal salt solution containing AgNO₃ was added to the salt precursor solution at a rate of 200 gm/hour with stirring at 2013 rpm. The metal salt solution was spray dried in air. The inlet temperature was 185° C., the outlet temperature was 90° C. and the feed rate was 2.7 kg/hr. The stabilized AgI nanoparticles had a water content of less than 5 weight %.

The stabilized AgI nanoparticle powder (0.32 grams) was dissolved in 199.7 grams DI water to prepare a solution. The AgI nanoparticle powder contained a nominal concentration of 6600 ppm silver as silver iodide. The concentration of silver in the final solution was calculated to be 11 ppm.

### Example 22

The method of Example 10 of US 2005/0013842 A1 was followed as described. Silver nitrate (0.127 grams) was dissolved in 75 mL of DI water to prepare a 0.01 M AgNO₃ solution. Polyacrylic acid (PAA, 2 grams) was dissolved in 48 mL of DI water to prepare a 4% w/w PAA solution. To 200 mL of DI water was added sodium borohydride (0.008 grams) to prepare a 1 mM solution. The 1 mM sodium borohydride solution (197 mL) was placed in a beaker with a stir bar. The beaker was immersed in an ice-water bath. The setup was placed on a stir plate. The 0.01M silver nitrate solution (2 mL) was mixed with 4% w/w PAA solution (1 mL), and cooled in an ice-water bath. The silver nitrate-PAA solution mixture was quickly added to the chilled 1 mM sodium borohydride solution with rapid stirring. An immediate brown-yellow discoloration was observed after mixing the solutions. The solution was mixed for 8 hours and then transferred to a clean amber jar for storage. Based on the amount of silver nitrate added the silver concentration of the final solution is calculated to be 11 ppm.

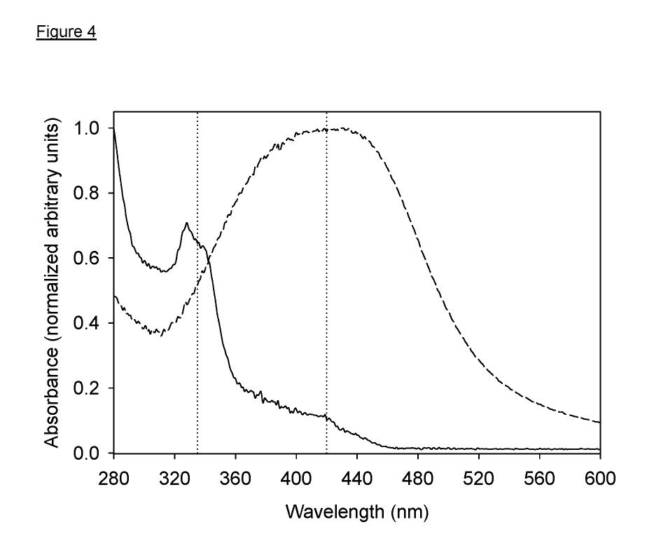

The UV-Vis spectrum of Ag-containing solution of this Example 22 was measured and is shown in FIG. 4, along with the UV-Vis spectrum of the aqueous AgI/PVP solution prepared in Synthetic Example 3. As can be seen from FIG. 4, the spectrum for the solution of this Example 22 had a broad peak centered at approximately 420 nm. In contrast, the main peak in the UV-Vis spectrum of the aqueous AgI/PVP dispersion of Synthetic Example 3 was centered at 330 nm. Based on Zang, Z. et al, this peak may be attributed to an interaction of silver present in ionic form (Ag⁺) with PVP in the aqueous solution. The differences in the spectra in FIG. 4 illustrate that the silver in the reactive mixtures and ophthalmic devices of the present invention is likely present in ionic form, whereas the silver present in the reactive mixtures of Examples 23A, B and E are present as Ag⁰.

### Examples 23A-B (Comparative)

The monomer components (other than the photoinitiator, Darocur 1173) listed in Table 9 were blended together in amber glass vials in the amounts listed in Table 9, and rolled on a jar roller for blending.

In Example 23A, a silver nitrate solution (0.025 gm AgNO₃, A.C.S. grade from Fisher dissolved in 54 mL anhydrous ethanol from Fisher) was used as the source of silver nitrate. In Example 23B, a silver nitrate solution (0.305 gm AgNO₃, A.C.S. grade from Fisher dissolved in 54 mL anhydrous ethanol from Fisher) was used as the source of silver nitrate.

TABLE 9

| | Comp. | | | | | |
|---|---|---|---|---|---|---|
| | 23A | 23B | 23C | 23D | 23E | 23F % |
| | % w/w | % w/w | % w/w | % w/w | % w/w | w/w |
| Macromer | 37.4 | 37.4 | 37.4 | 37.4 | 37.4 | 37.4 |
| TRIS | 15 | 15 | 15 | 15 | 15 | 15 |
| DMA | 22.5 | 22.5 | 22.5 | 22.5 | 22.5 | 22.5 |
| Darocur | 0.3 | 0.3 | 0.3 | 0.3 | 0.3 | 0.3 |
| 1173 | | | | | | |
| Ethanol | 24.8 | 24.8 | 24.3 | 18.9 | 24.8 | 24.8 |
| PAA | 0 | 0 | 0 | 0 | 0.022 | 0 |
| AgNO3 | 0.005 | 0.061 | 0 | 0 | 0.052 | 0 |
| AgI/K12 | 0 | 0 | 0.5 | 5.9 | 0 | 0 |
| powder* | | | | | | |
| Ag (ppm) | 35 | 343 | 37 | 241 | 449 | 0 |

*as formed in Synthetic Example 3

5 mL of each of the reactive mixtures from 23A and 23B were allowed to sit for 24 hours. The color of the reactive mixtures was measured quantitatively using the L*a*b* scale and the method described above. The color of the reactive mixtures was also evaluated subjectively under white fluorescent light. The results are disclosed in Table 10, below.

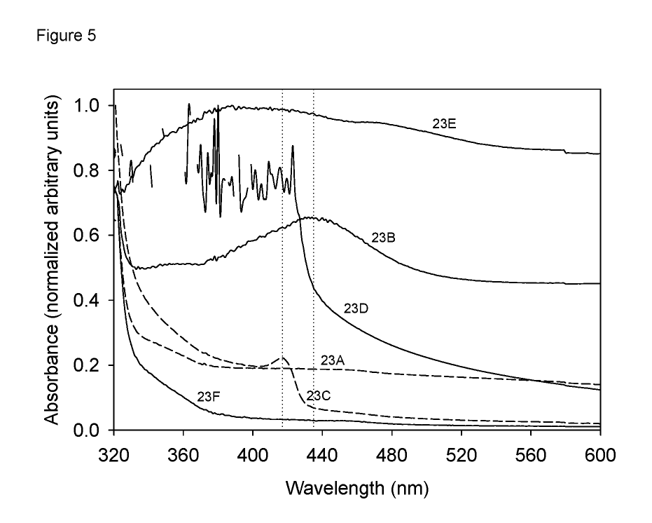

The UV-VIS spectra for the reactive mixtures of Examples 23 A and B were measured and are shown in FIG. 5.

The photoinitor (Darocur 1173) was added and each formulation was degassed for 5-7 minutes at 660-mmHg vacuum. The formulation was then transferred to a nitrogen glove box. Contact lenses were prepared using Zeonor front curves and Polypropylene back curves, which had been deoxygenated in the nitrogen glove box for at least 24 hours. A dose of 100 μL per lens cavity was used, and the frames holding the lens molds were placed under quartz plates. The lenses were cured for 60 minutes at room temperature under UV irradiation (bank of four parallel Philips TL09/20) bulbs.

After cure, the lens molds were opened manually, and the lenses were released in a jar containing 70:30 IPA:DI water mixture, utilizing ˜5 mL solution per lens. After at least 60 minutes, the lens molds were removed by tweezers, the solution was decanted, and the jar was filled with fresh 70:30 IPA:DI water mixture. The lenses were rolled on a jar roller, and after at least 60 minutes the solution was decanted, and the jar was filled with fresh DI water. The lenses were further rolled on a jar roller for at least 60 minutes, the solution was decanted, and the jar was filled with fresh DI water. Lenses were packaged in glass vials in 5 mL of phosphate buffered packaging solution, sealed with silicone stoppers and aluminum crimp caps, and autoclaved for 30 minutes at 122° C. Silver content of the lenses was measured using INAA and is reported in Table 9.

### Examples 23 C & D

Examples 23A and B were repeated, except that the stabilized AgI/PVP powder made in Synthetic Example 3 was added instead of the silver nitrate/ethanol solution. The color of the solution was measured as described in Examples 23 A & B, and are reported in Table 10. The UV-VIS spectra for the reactive mixtures of Example 23 C and D were measured and are shown in FIG. 5. Lenses were made as described in Examples 23A & B and packaged in glass vials in 5 mL of SSPS with 50 ppm methyl cellulose, sealed with silicone stoppers and aluminum crimp caps, and autoclaved for 30 minutes at 122° C. Silver content of the lenses was measured using INAA and is reported in Table 9.

### Example 23E

Example 23A was repeated, except that the silver source was 0.026 gm silver nitrate and 0.011 PAA dissolved in 11.25 gm of DMA instead of the silver nitrate/ethanol solution. The color of the solution was measured as described in Examples 23 A & B, and are reported in Table 10. The UV-VIS spectra for the reactive mixture of Example 23E was measured and is shown in FIG. 5. Lenses were made as described in Examples 23A & B. Silver content of the lenses was measured using INAA and is reported in Table 9.

### Example 23F

Example 23 A was repeated except that no silver was added.

TABLE 10

| | | | | Visual appearance |
|---|---|---|---|---|
| Ex# | L* | a* | b* | (color) |
| 23A | 85.08 | −0.45 | 2.58 | Brownish-yellow |
| 23B | 73.69 | −2.69 | 8.10 | Dark brownish-yellow |
| 23C | 89.40 | −1.91 | 2.77 | Slightly yellow |
| 23D | 86.28 | −3.84 | 10.85 | Yellow |
| 23E | 74.36 | −0.67 | 3.82 | Dark brown-yellow |
| 23F | 89.67 | −1.21 | 0.96 | Colorless |

FIG. 5 shows the comparison of the UV-Vis spectra for the reactive mixtures of Examples 23A-F. Example 23F, (the control formulation without silver) did not show any peaks in the region plotted. The reactive mixtures with low silver concentrations (Examples 23 A) also did not show any clear peaks. However, Example 23B shows a distinct peak at 435 nm, which according to US 2005/0013842 is confirmation of the presence of Ag⁰.

In the spectra for Example 23C a distinct transition was observed at 417 nm in the UV-Vis spectrum). The transition appeared to be present in the spectrum of the reactive mixture of Example 23D (having a target silver concentration of about 389 ppm), but the signal was noisy and close to saturation in that spectral region. Zhang, Z., Zhao, B., and Hu, L., Journal of Solid State Chemistry January 1996, 121, Issue 1, 5, 105-110. PVP Protective Mechanism of Ultrafine Silver Powder Synthesized by Chemical Reduction Processes obtained a spectral profile very similar to sample 23C, with an absorption shoulder at 420 nm, when they analyzed the UV-Vis spectrum of an AgI colloid. Furthermore, they discovered that upon reduction of the AgI colloid to Ag⁰ using sodium borohydride, the peak position and shape was very similar to that observed in the UV-Vis spectrum of sample 23B. Based on the data in scientific literature, the different shape and position of the peaks observed for Example 23C compared to the silver nitrate-based monomers of Example 23B is considered indicative of the presence of silver particles having different oxidation states.

### Examples 24A-F

The lenses formed in Examples 23A-F were tested for efficacy against staphylococcus aureus 031 using the procedure described in the test method section, above. The results are reported in Table 11, below.

TABLE 11

| | Log₁₀ Colony | | | |
|---|---|---|---|---|
| | Forming | | | |
| Lenses | Unit/lens or mL | Std. Dev. | % reduction | Log |
| from | (cfu/lens or | Of cfu/lens | to 23E | reduction to |
| Ex. # | mL) | or Ml | (control) | 23E (control) |
| 23F | 5.11 | 0.12 | Not | Not |
| | | | applicable | applicable |
| 23A | 5.92 | 0.22 | 0.0 | 0.0 |
| 23B | 5.87 | 0.11 | 0.0 | 0.0 |
| 23C | 3.07 | 0.04 | 99.1 | 2.0 |
| 23D | 3.26 | 0.03 | 98.6 | 1.9 |
| 23E | 4.95 | 1.07 | 0.0 | 0.2 |

Examples 23A and B have similar concentrations of silver in the lenses to Examples 23C and D, respectively. However, the antimicrobial activity data shows that lenses made from silver nitrate-containing monomers (23A,B, and E) did not demonstrate antimicrobial activity when compared to control lenses prepared in Example 23F. In contrast lenses prepared according to Examples 23C and D, which contain metal salt nanoparticles demonstrated at least a 1-log reduction compared to the control lenses.

### Example 25A

Example 23D was repeated, except that a visible light photoinitiator, CGI 819 was used, and the lenses were cured for 30 minutes at room temperature under visible irradiation (bank of four parallel Philips TL03/20) bulbs. The cured lenses were released, extracted, hydrated, packages and autoclaved as disclosed in Example 23D. The silver concentration, iodide concentration and color values were measured and are shown in Table 12, below. The silver concentration, iodide concentration and color values for the lenses of Example 23D (the same formulation made via UV curing) were also measured and are shown in Tables 12 and 13, below.

### Example 25B

Example 25 A was repeated, except that 2 wt % Norbloc was added to the formulation prior to curing, and the ethanol concentration was decreased by 2%. The silver concentration, iodide concentration and color values were measured after hydration and sterilization and are shown in Tables 12 and 13, below.

TABLE 12

| | Average | | | | |
|---|---|---|---|---|---|
| | [Ag] | Std. Dev. | Average [I] | Std. Dev. | Ag:I molar |
| Ex# | (ppm) | [Ag] (ppm) | (ppm) | [I] (ppm) | ratio |
| 25A | 485 | 15 | 587 | 18 | 1.00 |
| 25B | 471 | 4 | 564 | 6 | 0.98 |
| 23D | 241 | 30 | 160 | 52 | 1.8 |

The silver-to-iodide molar ratio (measured after hydration and sterilization) of lenses prepared according to Example 23D using UV light cure was observed to be approximately two. This data suggests that about half the silver content of the lenses was converted from silver iodide to silver of a different oxidation state during curing. It is believed that in Example 23D the UV light converted the AgI to Ag⁰ and I₂. Since the I₂ is soluble in IPA, it was removed during hydration. The expected silver-to-iodide molar ratio of lenses, based upon the silver iodide added to the reactive mixture was approximately one.

The silver-to-iodide molar ratio of lenses prepared in Examples 25A and B, using visible light cure was approximately one. Thus, the use of curing conditions outside the UV range is important in maintaining the antimicrobial metal salt, such as silver iodide, in its salt form.

TABLE 13

| Ex # | L* | a* | b* |
|---|---|---|---|
| 25A | 90.37 | −1.39 | 2.89 |
| 25B | 89.70 | −1.59 | 3.35 |
| 23D | 85.53 | −3.64 | 25.77 |

Based on the colorimetry data in Table 13, lenses of comparable silver concentrations prepared using visible light cure (Examples 25A & B), appeared significantly less yellow (lower b* values) than lenses prepared from Example 23D, which were UV light cured.

### Examples 26-28

A 100,000 ppm solution of PVP K12 was made in DI water. This solution (solution A) provided the base for making NaI and AgNO₃ solutions. Solutions of approximately 1500 ppm, 5000 ppm and 10000 ppm of each of NaI and AgNO₃ were made. Each solution was stirred until no visible particles were observed. A 20 mL portion of NaI solution was placed in a clean jar and magnetic stirrer was placed inside. The stirrer was set at 300 rpm and 20 ml. of AgNO₃ was added to the NaI solution at the rate shown in Table 14, below. All mixing was conducted at ambient temperature. The haze of the solution was subjectively assessed at the end of the listed addition time and results are reported in Table 14, below. The Example was repeated for each concentration and addition rate shown in Table 14.

TABLE 14

| Addn rate | Addn Time | Ex 26 | Ex 27 | Ex 28 |
|---|---|---|---|---|
| (ml/sec) | (sec) | 1500 ppm | 5000 ppm | 10,000 ppm |
| 20 | 1 | clear | Milky | Milky |
| 4 | 5 | clear | Mild haze | Milky |
| 2 | 10 | clear | Clear | Mild haze |
| 1 | 20 | clear | Clear | Clear |
| 0.67 | 30 | clear | Clear | Clear |

### Examples 29-31

Examples 26-28 were repeated, except that the NaI solution was added to the AgNO3 solution. The results are shown in Table 15, below.

TABLE 15

| Addn rate | Addn Time | Ex 29 | Ex 30 | Ex 31 |
|---|---|---|---|---|
| (ml/sec) | (sec) | 1500 ppm | 5000 ppm | 10,000 ppm |
| 20 | 1 | clear | Milky | Milky |
| 4 | 5 | clear | Milky | Milky |
| 2 | 10 | clear | Milky | Milky |
| 1 | 20 | Clear | Milky | Mild haze |
| 0.67 | 30 | Clear | Clear | Clear |

### Example 32

Example 31 was repeated, except that the metal agent and salt precursor solutions were mixed at room temperature on a jar roller for about ˜5 days, and then 20 ml of each solution was batch-wise mixed (poured together in about 1 second). The result was a clear solution comprising PVP-AgI complex.

### Examples 33-39

Approximately 10 mL of 700 ppm AgNO₃ solution was formed in PVP K12:DI water solution at the PVP concentrations shown in Table 16 (1% to 35% PVP K12 in DI water). Each AgNO₃ solution was dropwise-added to 10 mL of 1100 ppm NaI/DI solution (no PVP) with manual shaking to form dispersions. Example 33 was milky, the remaining Examples remained clear throughout addition of the AgNO₃. Particle size measurements were carried out on the resulting AgI dispersions using laser light scattering (Examples 33) and photon correlation spectrophotometry (Examples 35-39). Data is reported as z-average of the particle size distribution

TABLE 16

| | | Particle Size |
|---|---|---|
| Ex# | [PVP K12] (wt %) | (nm) |
| 33 | 0% | 10600 |
| 34 | 1% | 270 |
| 35 | 2% | 40 |
| 36 | 10% | 540 |
| 37 | 15% | 400 |
| 38 | 25% | 40 |
| 39 | 35% | 20 |

The data from Table 16 is shown graphically in FIG. 5. The data in Table 16 clearly shows that the presence of PVP during metal salt formation decreases particle size substantially (at least two orders of magnitude).

### Examples 40-44

Example 34 was repeated, except the dispersing agents listed in Table 17 were used instead of PVP, and at the concentrations listed in Table 17. Particle size measurements were carried out on the resulting AgI dispersions using laser light scattering (40, 41 and 43) and photon correlation spectrophotometry (42, 44). Data is reported as z-average of the particle size distribution.

TABLE 17

| | | Particle Size |
|---|---|---|
| Ex# | Dispersing agent | (nm) |
| 40 | 5% PAA 2K | 2760 |
| 41 | 5% PEO 10K | 7020 |
| 42 | 10% PEO 10K | 475 |
| 43 | GLYCERIN | 6380 |
| 44 | PVA 120K | 470 |

### Example 45

The components shown in Table 18 below were blended together in amber glass vials in the amounts listed in Table 18, and rolled on a jar roller. The mixture was dispensed into contact lens molds (Zeonor front and back curve molds) and cured under the following conditions: 2.8+/−0.5% O₂; visible light cure (Philips TL03 lamps); Intensity profile: 1+/−0.5 mW/cm² (10-60 sec) at 25° C., 5.5+/−0.5 mW/cm² (304-600 sec) at 80+/−5° C. The lenses were hydrated in IPA/water mixtures, packaged in individual polypropylene blister packs in 950 microliters of SSPS with 50 ppm methyl cellulose, and autoclaved for 18 minutes at 124° C.

| | Ex. 45 | |
|---|---|---|
| Component | (% w/w) | Control (% w/w) |
| Norbloc | 0.9 | 0.9 |
| CGI 819 | 0.14 | 0.14 |
| mPDMS 1000 | 13.2 | 13.2 |
| DMA | 18.6 | 18.6 |
| HEMA | 5.10 | 5.1 |
| EGDMA | 0.45 | 0.45 |
| SiMMA | 18 | 18 |
| Blue Hema | 0.01 | 0.01 |
| PVP K90 | 3.6 | 3.6 |
| t-amyl alcohol | 29 | 29 |
| PVP K12 | 5.5 | 11 |
| Agl particles | 5.5 | 0 |
| TOTAL | 100 | 100 |

Twelve lenses formed in this Example 45 were tested for efficacy against staphylococcus aureus 031 using the procedure described in the test method section, above. The control lenses were made per the method of Example 45, but did not contain silver iodide nanoparticles. The log reduction (vs. control) of the silver containing lenses was determined to be 3.3±0.2 (average+/−standard deviation).

### Example 46

The lenses of Example 45 were worn by 30 human patients (all current contact lens wearers) in a double masked, contralateral clinical trial with the control lenses of Example 45. The patients wore the lenses for 14 days, in daily wear modality, used OptiFree RepleniSH and were instructed to rub their lenses during lens cleaning and disinfecting. The lenses of Example 45 contained approximately 10 μg of silver at baseline.

The worn lenses from the 26 patients who completed the study were collected at the end of the 14 day wear period and tested for silver content by INAA. From the INAA data the mean rate of silver release was calculated to be 0.5 μg per day. The lenses were also tested for activity against S. aureus using the method described in the test method section, above. The log reduction (vs. worn control) of the lens of Example 45 was determined to be 3.4±1.2 (average+/−standard deviation).

1-20. (canceled)

21. A process comprising the steps of
(a) dissolving in a solvent at least one salt precursor, optionally with at least one component of a reactive polymer mixture to form a salt precursor mixture;
(b) forming a dispersing agent-metal agent complex by dissolving in a solvent at least one metal agent and at least one dispersing agent, optionally with at least one reactive component to form a metal agent mixture, wherein said solvents and components may be the same or different;
(c) mixing said salt precursor mixture and said metal agent mixture under particle forming conditions to form a particle-containing mixture comprising at least one antimicrobial metal salt, [M^{q+}]ₐ[X^{z-}]_{b};
(d) optionally mixing additional reactive components with said particle containing mixture to form a particle-containing reaction mixture; with the proviso that where no reactive components are included in steps (a) and (b), at least one reactive component is added in step (d); and
(e) reacting said particle containing reactive mixture to form an antimicrobial polymeric article under reaction conditions sufficient to maintain at least about 90% of M from said metal agent added in step (c) in said polymeric article as M^{q+}.

22. The process of claim 21 wherein the at least one reactive component optionally used in step (a) or (b) is a non-reactive with the metal agent.

23. The process of claim 21 wherein reactive components which are reactive with the metal agent are added to the particle containing reactive mixture in mixing step (d).

24. The process of claim 21 wherein said dispersing agent is selected from the group consisting of hydroxyalkylmethylcellulose polymers, polyvinyl alcohol, polyvinyl pyrrolidone, polyethylene oxide, starch, pectin, polyacrylamide, gelatin, polyacrylic acid, organoalkoxysilanes 3-aminopropyltriethoxysilane, methyltriethoxysilane, phenyltrimethoxysilane, vinyltriethoxysilane, and 3-glycidoxypropyltrimethoxysilane, boric acid ester of glycerin and mixtures thereof.

25. The process of claim 21 wherein said dispersing agent is selected from the group consisting of hydroxyalkylmethylcellulose polymers, polyvinyl alcohol, polyvinyl pyrrolidone, polyethylene oxide, gelatin and polyacrylic acid, boric acid ester of glycerin and mixtures thereof.

26. The process of claim 21 wherein said dispersing agent is selected from the group consisting of hydroxypropylmethylcellulose, polyvinyl alcohol, polyvinyl pyrrolidone, polyethylene oxide, gelatin and polyacrylic acid, and mixtures thereof.

27. The process of claim 21 wherein said dispersing agent is selected from the group consisting of polyvinyl alcohol, polyvinyl pyrrolidone, polyethylene oxide, and polyacrylic acid, and mixtures thereof.

28. The process of claim 27 wherein said dispersing agent has a molecular weight of less than about 2,000,000.

29. The process of claim 27 wherein said dispersing agent has a molecular weight between about 20,000 and about 1,500,000.

30. The process of claim 21 wherein said metal agent mixture comprises up to about 40 weight % of the dispersing agent.

31. The process of claim 21 wherein said metal agent mixture comprises between 0.01 weight % and about 30 weight % of the dispersing agent.

32. The process of claim 21 wherein said salt precursor mixture comprises up to about 10 weight % of the salt precursor.

33. The process of claim 21 wherein said metal salt precursor mixture comprises the salt precursor in a molar excess relative to said metal agent.

34. The process of claim 21 wherein said metal agent mixture comprises up to about 10 weight % of the metal agent.

35. The process of claim 21 wherein said solvent is removed from particle containing mixture prior to step (d).

36-39. (canceled)

40. The process of claim 21 wherein the salt precursor is sodium iodide.

41. The process of claim 21 wherein the metal agent is silver acetate.

42. The process of claim 21 wherein the dispersing agent is polyvinyl pyrrolidone.

43. The process of claim 21 wherein the salt percursor is sodium iodide, the metal agent is silver acetate, and the dispersing agent is polyvinyl pyrrolidone.
